SEQUENCE LISTING (14 sequences)

SEQ ID NO: 1 - protein, 3 aa, Artificial Sequence
  Exemplary linker sequence
GGG

SEQ ID NO: 2 - protein, 5 aa, Artificial Sequence
  Exemplary linker sequence
DGGGS

SEQ ID NO: 3 - protein, 5 aa, Artificial Sequence
  Exemplary linker sequence
TGEKP

SEQ ID NO: 4 - protein, 4 aa, Artificial Sequence
  Exemplary linker sequence
GGRR

SEQ ID NO: 5 - protein, 5 aa, Artificial Sequence
  Exemplary linker sequence
GGGGS

SEQ ID NO: 6 - protein, 14 aa, Artificial Sequence
  Exemplary linker sequence
EGKSSGSGSESKVD

SEQ ID NO: 7 - protein, 18 aa, Artificial Sequence
  Exemplary linker sequence
KESGSVSSEQLAQFRSLD

SEQ ID NO: 8 - protein, 8 aa, Artificial Sequence
  Exemplary linker sequence
GGRRGGGS

SEQ ID NO: 9 - protein, 9 aa, Artificial Sequence
  Exemplary linker sequence
LRQRDGERP

SEQ ID NO: 10 - protein, 12 aa, Artificial Sequence
  Exemplary linker sequence
LRQKDGGGSERP

SEQ ID NO: 11 - protein, 16 aa, Artificial Sequence
  Exemplary linker sequence
LRQKDGGGSGGGSERP

SEQ ID NO: 12 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 13 - protein, 7 aa, Artificial Sequence
  Cleavage sequence by TEV protease
ENLYFQG

SEQ ID NO: 14 - protein, 7 aa, Artificial Sequence
  Cleavage sequence by TEV protease
ENLYFQS

(19) United States
(12) Patent Application Publication
(10) Pub. No.: US 2019/0169597 A1
(43) Pub. Date: Jun. 6, 2019

(54) GENOME EDITING ENHANCERS

(71) Applicant: bluebird bio, Inc., Cambridge, MA (US)

(72) Inventors: Sasha ASTRAKHAN, Seattle, WA (US); Garrett C. HEFFNER, San Francisco, CA (US)

(73) Assignee: bluebird bio, Inc., Cambridge, MA (US)

(21) Appl. No.: 16/325,013

(22) PCT Filed: Aug. 18, 2017
(86) PCT No.: PCT/US2017/047535

§ 371 Date: Feb. 12, 2019

Related U.S. Application Data
(60) Provisional application No. 62/377,357, filed on Aug. 19, 2016.

(51) Int. Cl. C12N 15/10; C12N 9/22; C12N 15/11; C12N 15/90; A61K 35/17

(52) U.S. Cl. CPC C12N 15/102; C12N 9/22; C12N 15/11; C12N 2310/20; A61K 35/17; C12N 2800/80; C12N 15/907

(57) ABSTRACT

The invention provides improved compositions for gene therapy compositions for treatment, prevention, or amelioration of numerous diseases, disorders, and conditions.

### CROSS REFERENCE TO RELATED APPLICATIONS

This application claims the benefit under 35 U.S.C. § 119(e) of U.S. Provisional Application No. 62/377,357, filed Aug. 19, 2016, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_074_01WO_ST25.txt. The text file is 4 KB, was created on Aug. 18, 2017, and is being submitted electronically via EFS-Web, concurrent with the filing of the specification.

### BACKGROUND

### Technical Field

The present invention generally relates, in part, to improved gene therapy compositions and methods of making the same. More particularly, the invention relates to improved genome editing compositions and gene therapies and methods of making the same.

### Description of the Related Art

Mutations in 3000 human genes have already been linked to disease phenotypes (www.omim.org/statistics/geneMap), and more disease relevant genetic variations are being uncovered at a staggeringly rapid pace. However, despite valid therapeutic hypotheses and strong efforts in drug development, only a mere handful of successes exists in using small molecules to treat diseases with strong genetic contributions. The vast potential of emerging genome editing strategies based on programmable nucleases such as meganucleases, zinc finger nucleases, transcription activator-like effector nucleases and the clustered regularly interspaced short palindromic repeat (CRISPR)-associated nuclease Cas9 for the treatment of monogenic, highly penetrant diseases has yet to be realized. Particular hurdles to implementing nuclease-based genome editing strategies include, but are not limited to low genome editing efficiencies, nuclease specificity, and delivery challenges. The current state of the art for most genome editing strategies falls short in some or all of these criteria.

### BRIEF SUMMARY

The invention generally relates, in part, to improved genome editing compositions and methods of using the same to develop safer and more efficacious gene therapies.

In various embodiments, the present invention contemplates, in part, a composition comprising a population of cells, a genome editing enhancer, and an engineered nuclease, and/or optionally, an mRNA encoding the engineered nuclease.

In various embodiments, the present invention contemplates, in part, a composition comprising a population of cells, a genome editing enhancer, a donor repair template, and an engineered nuclease, and/or optionally, an mRNA encoding the engineered nuclease.

In particular embodiments, the population of cells comprises stem cells.

In some embodiments, the population of cells comprises hematopoietic cells.

In certain embodiments, the population of cells comprises CD34⁺ cells, CD133⁺ cells, CD34⁺CD133⁺ cells, or CD34⁺CD38^{Lo}CD90⁺CD45RA⁻ cells.

In particular embodiments, the population of cells comprises immune effector cells.

In additional embodiments, the population of cells comprises CD3⁺, CD4⁺, CD8⁺ cells, or a combination thereof.

In certain embodiments, the population of cells comprises T cells.

In further embodiments, the population of cells comprises cytotoxic T lymphocytes (CTLs), a tumor infiltrating lymphocytes (TILs), or a helper T cells.

In some embodiments, the source of the cells is peripheral blood mononuclear cells, bone marrow, lymph nodes tissue, cord blood, thymus issue, tissue from a site of infection, ascites, pleural effusion, spleen tissue, or tumors.

In various embodiments, the present invention contemplates, in part, a composition comprising a genome editing enhancer, and an engineered nuclease, and/or optionally, an mRNA encoding the engineered nuclease.

In various embodiments, the present invention contemplates, in part, a composition comprising a genome editing enhancer, a donor repair template, and an engineered nuclease, and/or optionally, an mRNA encoding the engineered nuclease.

In additional embodiments, the genome editing enhancer is a DNA intercalator.

In particular embodiments, the genome editing enhancer is selected from the group consisting of: a monofunctional DNA intercalator, a bifunctional DNA intercalator, or a polyfunctional DNA intercalator.

In additional embodiments, the genome editing enhancer is selected from the group consisting of: acridines, anthracyclines, alkaloids, coumarins, and phenanthridines.

In particular embodiments, the genome editing enhancer is selected from the group consisting of: 1,8-naphthalimide, 4′6-diamidino-α-phenylindole, acridines, acridine orange, acriflavine, acronycine, actinodaphnidine, aminacrine, amsacrine, anthracycline, anthramycin, anthrapyrazole, benzophenanthridine alkaloids, berbamine, berberine, berberrubine, bleomycin, BOBO-1, BOBO-3, boldine, BO-PRO-1, BO-PRO-3, bublocapnine, camptothecin, cassythine, chartreusin, chloroquine, chromomycin, cinchonidine, cinchonine, coptisine, coralyne, coumarin, cryptolepine, dactinomycin, DAPI, daunorubicin, dicentrine, dictamine, distamycin, doxorubicin, ellipticine, emetine, ethacridine, ethidium, evolitrine, fagarine, fagaronine, fluorcoumanin, GelStar, gentamicin, glaucine, harmaline, harmine, harmine, hedamycin, hexidium, Hoechst 33258, Hoechst 33342, homidium, hycanthone, imidazoacridinone, an indazole analog, iodide, isocorydine, isoquinoline alkaloids, jatrorrhizine, JOJO-1, JO-PRO-1, kinetin riboside, kokusainine, lobeline, LOLO-1, LO-PRO-1, lucanthone, masculine, matadine, mepacrine, a metallo-intercalator, mithramycin, mitoxantrone, neocryptolepine, netropsin, nitidine, nitracrine, nogalamycin, norharman, OliGreen, palmatine, phenanthridine, PicoGreen, pirarubicin, polypyridyls, POPO-1, POPO-3, PO-PRO-1, PO-PRO-3, proflavine, propidium, psoralen, quinacrine, quinidine, quinine, quinoxalines, RiboGreen, a rhodium based intercalator, a ruthenium based intercalator, sanguinarine, serpentine, skimmianine, streptomycin, SYBR DX, SYBR Gold, SYBR Green I, SYBR Green II, SYTO-11, SYTO-12, SYTO-13, SYTO-14, SYTO-15, SYTO-16, SYTO-17, SYTO-20, SYTO-21, SYTO-22, SYTO-23, SYTO-24, SYTO-25, SYTO-40, SYTO-41, SYTO-42, SYTO-43, SYTO-44, SYTO-45, SYTO-59, SYTO-60, SYTO-61, SYTO-62, SYTO-63, SYTO-64, SYTO-80, SYTO-81, SYTO-82, SYTO-83, SYTO-84, SYTO-85, SYTOX blue, SYTOX green, SYTOX orange, tacrine, thalidomide, thiazole orange, tilorone, TO-PRO-1, TO-PRO-3, TO-PRO-5, TOTO-1, TOTO-3, usambarensine, YO-PRO-1, YO-PRO-3, YOYO-1, YOYO-3, and analogs and derivatives thereof.

In certain embodiments, the genome editing enhancer is selected from the group consisting of: tilorone, aminacrine, homidium bromide (ethidium bromide), harmine, hycanthone, daunorubicin, sanguinarine sulfate, kinetin riboside, ethacridine lactate, and cyclohexamide.

In further embodiments, the genome editing enhancer is selected from the group consisting of: tilorone, aminacrine, homidium bromide (ethidium bromide), and harmine.

In particular embodiments, the genome editing enhancer is an acridine or diacridine.

In some embodiments, the genome editing enhancer is aminacrine (9-aminoacridine).

In various embodiments, the present invention contemplates, in part, a composition comprising a cell, an acridine, and an engineered nuclease, and/or optionally, an mRNA encoding the engineered nuclease.

In particular embodiments, the present invention contemplates, in part, a composition comprising a cell, an acridine, a donor repair template, and an engineered nuclease, and/or optionally, an mRNA encoding the engineered nuclease.

In additional embodiments, the present invention contemplates, in part, a composition comprising a cell, 9-aminoacridine, and an engineered nuclease, and/or optionally, an mRNA encoding the engineered nuclease.

In certain embodiments, the present invention contemplates, in part, a composition comprising a cell, 9-aminoacridine, a donor repair template, and an engineered nuclease, and/or optionally, an mRNA encoding the engineered nuclease.

In some embodiments, the present invention contemplates, in part, a composition comprising an acridine, and an engineered nuclease, and/or optionally, an mRNA encoding the engineered nuclease.

In further embodiments, the present invention contemplates, in part, a composition comprising an acridine, a donor repair template, and an engineered nuclease, and/or optionally, an mRNA encoding the engineered nuclease.

In particular embodiments, the present invention contemplates, in part, a composition comprising 9-aminoacridine, and an engineered nuclease, and/or optionally, an mRNA encoding the engineered nuclease.

In various embodiments, the present invention contemplates, in part, a composition comprising 9-aminoacridine, a donor repair template, and an engineered nuclease, and/or optionally, an mRNA encoding the engineered nuclease.

In particular embodiments, the engineered nuclease is selected from the group consisting of: a meganuclease, a megaTAL, a TALEN, a ZFN, or a CRISPR/Cas nuclease.

In particular embodiments, the meganuclease is engineered from an LAGLIDADG homing endonuclease (LHE) selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

In additional embodiments, the meganuclease is engineered from an LHE selected from the group consisting of: I-CpaMI, I-HjeMI, I-OnuI, I-PanMI, and SmaMI.

In certain embodiments, the meganuclease is engineered from an I-OnuI LHE.

In some embodiments, the megaTAL comprises a TALE DNA binding domain and an engineered meganuclease.

In particular embodiments, the TALE binding domain comprises about 9.5 TALE repeat units to about 11.5 TALE repeat units.

In some embodiments, the meganuclease is engineered from an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

In certain embodiments, the meganuclease is engineered from an LHE selected from the group consisting of: I-CpaMI, I-HjeMI, I-OnuI, I-PanMI, and SmaMI.

In particular embodiments, the meganuclease is engineered from an I-OnuI LHE.

In further embodiments, the TALEN comprises a TALE DNA binding domain and an endonuclease domain or half-domain.

In particular embodiments, the TALE DNA binding domain comprises about 9.5 TALE repeat units to about 11.5 TALE repeat units.

In additional embodiments, the endonuclease domain is isolated from a type-II restriction endonuclease.

In particular embodiments, the endonuclease domain is isolated from FokI.

In further embodiments, the ZFN comprises a zinc finger DNA binding domain and an endonuclease domain or half-domain.

In some embodiments, the zinc finger DNA binding domain comprises 2, 3, 4, 5, 6, 7, or 8 zinc finger motifs.

In additional embodiments, the ZFN comprises a TALE binding domain.

In particular embodiments, the TALE DNA binding domain comprises about 9.5 TALE repeat units to about 11.5 TALE repeat units.

In some embodiments, the endonuclease domain is isolated from a type-II restriction endonuclease.

In certain embodiments, the endonuclease domain is isolated from FokI.

In further embodiments, the engineered nuclease comprises a CRISPR/Cas nuclease.

In particular embodiments, the Cas nuclease is Cas9 or Cpf1.

In additional embodiments, the Cas nuclease further comprises one or more TALE DNA binding domains.

In some embodiments, the composition further comprises a tracrRNA, and one or more crRNAs that target a protospacer sequence in the genome of the cell.

In particular embodiments, the composition further comprises one or more sgRNAs that target a protospacer sequence in the genome of the cell.

In additional embodiments, the engineered nuclease comprises an end-processing enzymatic activity.

In particular embodiments, the end-processing enzymatic activity is 5-3′ exonuclease, 5-3′ alkaline exonuclease, 3-5′exonuclease, 5′ flap endonuclease, helicase or template-independent DNA polymerases activity.

In further embodiments, the end-processing enzymatic activity is 3-5′exonuclease activity of Trex2 or a biologically active fragment thereof.

In certain embodiments, the composition further comprises an end-processing enzyme, or an mRNA encoding the end-processing enzyme.

In additional embodiments, the end-processing enzyme exhibits 5-3′ exonuclease, 5-3′ alkaline exonuclease, 3-5′exonuclease, 5′ flap endonuclease, helicase or template-independent DNA polymerases activity.

In some embodiments, the end-processing enzyme comprises Trex2 or a biologically active fragment thereof.

In particular embodiments, the composition comprises a donor repair template that encodes: β globin, δ globin, γ globin, BCL11A, KLF1, CCR5, CXCR4, PPP1R12C (AAVS1), HPRT, albumin, Factor VIII, Factor IX, LRRK2, Htt, SOD1, C9orf72, TARDBP, FUS, RHO, CFTR, SFTPB, TRAC, TRBC, PD1, CTLA-4, HLA A, HLA B, HLA C, HLA-DP, HLA-DQ, HLA-DR, LMP7, TAP 1, TAP2, TAPBP, CIITA, DMD, GR, IL2RG, Rag-1, RFX5, FAD2, FAD3, ZP15, KASII, MDH, EPSPS, or a fragment thereof.

In certain embodiments, the composition comprises a donor repair template that encodes a bispecific T cell engager (BiTE) molecule; a hormone; a cytokine (e.g., IL-2, insulin, IFN-γ, IL-7, IL-21, IL-10, IL-12, IL-15, and TNF-α), a chemokine (e.g., MIP-1α, MIP-1β, MCP-1, MCP-3, and RANTES), a cytotoxin (e.g., Perforin, Granzyme A, and Granzyme B), a cytokine receptor (e.g., an IL-2 receptor, an IL-7 receptor, an IL-12 receptor, an IL-15 receptor, and an IL-21 receptor), or an engineered antigen receptor.

In additional embodiments, the composition comprises a donor repair template that encodes an engineered T cell receptor (TCR), a chimeric antigen receptor (CAR), a Daric receptor or components thereof, or a chimeric cytokine receptor.

In various embodiments, the present invention contemplates, in part, a method of increasing genome editing in a population of cells comprising: introducing an engineered nuclease into a population of cells; and contacting the population of cells with a genome editing enhancer, wherein expression of the engineered nuclease in the presence of the genome editing enhancer increases the frequency of genome editing in the population of cells.

In various embodiments, the present invention contemplates, in part, a method of increasing homology directed repair (HDR) in a population of cells comprising: contacting the population of cells with a genome editing enhancer; introducing an engineered nuclease to generate a double-strand break (DSB) at a target site; and introducing a donor repair template into the population of cells; wherein expression of the engineered nuclease in the presence of the genome editing enhancer and the donor repair template increases the frequency of incorporation of the donor repair template at the target site by homology directed repair (HDR).

In various embodiments, the present invention contemplates, in part, a method of increasing non-homologous end joining (NHEJ) in a population of cells comprising: contacting the population of cells with a genome editing enhancer; introducing an engineered nuclease to generate a double-strand break (DSB) at a target site; introducing an engineered nuclease into a population of cells; and wherein expression of the engineered nuclease in the presence of the genome editing enhancer increases the frequency of NHEJ at the target site.

In further embodiments, the cell is a hematopoietic cell.

In additional embodiments, the cell is an immune effector cell.

In some embodiments, the cell is CD3⁺, CD4⁺, CD8⁺, or a combination thereof.

In particular embodiments, the cell is a T cell.

In additional embodiments, the cell is a cytotoxic T lymphocyte (CTL), a tumor infiltrating lymphocyte (TIL), or a helper T cell.

In further embodiments, the source of the cell is peripheral blood mononuclear cells, bone marrow, lymph nodes tissue, cord blood, thymus issue, tissue from a site of infection, ascites, pleural effusion, spleen tissue, or tumors.

In certain embodiments, the cell is a hematopoietic stem cell or hematopoietic progenitor cell.

In further embodiments, the cell is a CD34⁺ cell.

In particular embodiments, the cell is a CD133⁺ cell.

In some embodiments, the cell is a CD34⁺CD38LoCD90⁺CD45RA⁻ cell.

In particular embodiments, the genome editing enhancer is a DNA intercalator.

In additional embodiments, the genome editing enhancer is selected from the group consisting of: a monofunctional DNA intercalator, a bifunctional DNA intercalator, or a polyfunctional DNA intercalator.

In certain embodiments, the genome editing enhancer is selected from the group consisting of: acridines, anthracyclines, alkaloids, coumarins, and phenanthridines.

In further embodiments, the genome editing enhancer is selected from the group consisting of: 1,8-naphthalimide, 4′6-diamidino-α-phenylindole, acridines, acridine orange, acriflavine, acronycine, actinodaphnidine, aminacrine, amsacrine, anthracycline, anthramycin, anthrapyrazole, benzophenanthridine alkaloids, berbamine, berberine, berberrubine, bleomycin, BOBO-1, BOBO-3, boldine, BO-PRO-1, BO-PRO-3, bublocapnine, camptothecin, cassythine, chartreusin, chloroquine, chromomycin, cinchonidine, cinchonine, coptisine, coralyne, coumarin, cryptolepine, dactinomycin, DAPI, daunorubicin, dicentrine, dictamine, distamycin, doxorubicin, ellipticine, emetine, ethacridine, ethidium, evolitrine, fagarine, fagaronine, fluorcoumanin, GelStar, gentamicin, glaucine, harmaline, harmine, harmine, hedamycin, hexidium, Hoechst 33258, Hoechst 33342, homidium, hycanthone, imidazoacridinone, an indazole analog, iodide, isocorydine, isoquinoline alkaloids, jatrorrhizine, JOJO-1, JO-PRO-1, kinetin riboside, kokusainine, lobeline, LOLO-1, LO-PRO-1, lucanthone, masculine, matadine, mepacrine, a metallo-intercalator, mithramycin, mitoxantrone, neocryptolepine, netropsin, nitidine, nitracrine, nogalamycin, norharman, OliGreen, palmatine, phenanthridine, PicoGreen, pirarubicin, polypyridyls, POPO-1, POPO-3, PO-PRO-1, PO-PRO-3, proflavine, propidium, psoralen, quinacrine, quinidine, quinine, quinoxalines, RiboGreen, a rhodium based intercalator, a ruthenium based intercalator, sanguinarine, serpentine, skimmianine, streptomycin, SYBR DX, SYBR Gold, SYBR Green I, SYBR Green II, SYTO-11, SYTO-12, SYTO-13, SYTO-14, SYTO-15, SYTO-16, SYTO-17, SYTO-20, SYTO-21, SYTO-22, SYTO-23, SYTO-24, SYTO-25, SYTO-40, SYTO-41, SYTO-42, SYTO-43, SYTO-44, SYTO-45, SYTO-59, SYTO-60, SYTO-61, SYTO-62, SYTO-63, SYTO-64, SYTO-80, SYTO-81, SYTO-82, SYTO-83, SYTO-84, SYTO-85, SYTOX blue, SYTOX green, SYTOX orange, tacrine, thalidomide, thiazole orange, tilorone, TO-PRO-1, TO-PRO-3, TO-PRO-5, TOTO-1, TOTO-3, usambarensine, YO-PRO-1, YO-PRO-3, YOYO-1, YOYO-3, and analogs and derivatives thereof.

In certain embodiments, the genome editing enhancer is selected from the group consisting of: tilorone, aminacrine, homidium bromide (ethidium bromide), harmine, hycanthone, daunorubicin, sanguinarine sulfate, kinetin riboside, ethacridine lactate, and cyclohexamide.

In additional embodiments, the genome editing enhancer is selected from the group consisting of: tilorone, aminacrine, homidium bromide (ethidium bromide), and harmine.

In particular embodiments, the genome editing enhancer is an acridine or diacridine.

In particular embodiments, the genome editing enhancer is aminacrine (9-aminoacridine).

In further embodiments, the engineered nuclease is selected from the group consisting of: a meganuclease, a megaTAL, a TALEN, a ZFN, or a CRISPR/Cas nuclease.

In additional embodiments, the meganuclease is engineered from an LAGLIDADG homing endonuclease (LHE) selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

In certain embodiments, the meganuclease is engineered from an LHE selected from the group consisting of: I-CpaMI, I-HjeMI, I-OnuI, I-PanMI, and SmaMI.

In some embodiments, the meganuclease is engineered from an I-OnuI LHE.

In further embodiments, the megaTAL comprises a TALE DNA binding domain and an engineered meganuclease.

In particular embodiments, the TALE binding domain comprises about 9.5 TALE repeat units to about 11.5 TALE repeat units.

In additional embodiments, the meganuclease is engineered from an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

In certain embodiments, the meganuclease is engineered from an LHE selected from the group consisting of: I-CpaMI, I-HjeMI, I-OnuI, I-PanMI, and SmaMI.

In particular embodiments, the meganuclease is engineered from an I-OnuI LHE.

In some embodiments, the TALEN comprises a TALE DNA binding domain and an endonuclease domain or half-domain.

In additional embodiments, the TALE DNA binding domain comprises about 9.5 TALE repeat units to about 11.5 TALE repeat units.

In particular embodiments, the endonuclease domain is isolated from a type-II restriction endonuclease.

In further embodiments, the endonuclease domain is isolated from FokI.

In certain embodiments, the ZFN comprises a zinc finger DNA binding domain and an endonuclease domain or half-domain.

In some embodiments, the zinc finger DNA binding domain comprises 2, 3, 4, 5, 6, 7, or 8 zinc finger motifs.

In certain embodiments, the ZFN comprises a TALE binding domain.

In additional embodiments, the TALE DNA binding domain comprises about 9.5 TALE repeat units to about 11.5 TALE repeat units.

In further embodiments, the endonuclease domain is isolated from a type-II restriction endonuclease.

In particular embodiments, the endonuclease domain is isolated from FokI.

In additional embodiments, the engineered nuclease comprises a CRISPR/Cas nuclease.

In some embodiments, the Cas nuclease is Cas9 or Cpf1.

In certain embodiments, the Cas nuclease further comprises one or more TALE DNA binding domains.

In particular embodiments, the composition further comprises a tracrRNA, and one or more crRNAs that target a protospacer sequence in the genome of the cell.

In additional embodiments, the composition further comprises one or more sgRNAs that target a protospacer sequence in the genome of the cell.

In further embodiments, the engineered nuclease comprises an end-processing enzymatic activity.

In some embodiments, the end-processing enzymatic activity is 5-3′ exonuclease, 5-3′ alkaline exonuclease, 3-5′exonuclease, 5′ flap endonuclease, helicase or template-independent DNA polymerases activity.

In particular embodiments, the end-processing enzymatic activity is 3-5′exonuclease activity of Trex2 or a biologically active fragment thereof.

In particular embodiments, the composition further comprises an end-processing enzyme, or an mRNA encoding the end-processing enzyme.

In particular embodiments, the end-processing enzyme exhibits 5-3′ exonuclease, 5-3′ alkaline exonuclease, 3-5′exonuclease, 5′ flap endonuclease, helicase or template-independent DNA polymerases activity.

In additional embodiments, the end-processing enzyme comprises Trex2 or a biologically active fragment thereof.

In certain embodiments, the method comprises a donor repair template that encodes: β globin, δ globin, γ globin, BCL11A, KLF1, CCR5, CXCR4, PPP1R12C (AAVS1), HPRT, albumin, Factor VIII, Factor IX, LRRK2, Htt, SOD1, C9orf72, TARDBP, FUS, RHO, CFTR, SFTPB, TRAC, TRBC, PD1, CTLA-4, HLA A, HLA B, HLA C, HLA-DP, HLA-DQ, HLA-DR, LMP7, TAP 1, TAP2, TAPBP, CIITA, DMD, GR, IL2RG, Rag-1, RFX5, FAD2, FAD3, ZP15, KASII, MDH, EPSPS, or a fragment thereof.

In further embodiments, the method comprises a donor repair template that encodes: a bispecific T cell engager (BiTE) molecule; a hormone; a cytokine (e.g., IL-2, insulin, IFN-γ, IL-7, IL-21, IL-10, IL-12, IL-15, and TNF-α), a chemokine (e.g., MIP-1α, MIP-1β, MCP-1, MCP-3, and RANTES), a cytotoxin (e.g., Perforin, Granzyme A, and Granzyme B), a cytokine receptor (e.g., an IL-2 receptor, an IL-7 receptor, an IL-12 receptor, an IL-15 receptor, and an IL-21 receptor), or an engineered antigen receptor.

In particular embodiments, the method comprises a donor repair template that encodes: an engineered T cell receptor (TCR), a chimeric antigen receptor (CAR), a Daric receptor or components thereof, or a chimeric cytokine receptor.

In various embodiments, the present invention contemplates, in part, a cell produced by a method contemplated herein.

In various embodiments, the present invention contemplates, in part, a composition comprising a cell contemplated herein.

In various embodiments, the present invention contemplates, in part, a pharmaceutical composition comprising a pharmaceutically acceptable carrier and a cell contemplated herein.

### BRIEF DESCRIPTION OF SEVERAL VIEWS OF THE DRAWINGS

FIG. 1 shows the analytic workflow of flow cytometry data.

FIG. 2A shows a plot of the compounds in rank order according to effect on cell yield.

FIG. 2B shows a plot of the compounds in rank order according to the frequency of CD3-negative cells.

FIG. 2C shows a plot of the compounds according to the frequency of CD3-negative cells as a function of cell yield.

FIG. 3A shows a dose response curve of the compounds in an assay to measure non-homologous end joining editing efficiency in primary T cells at 37° C.

FIG. 3B shows a dose response curve of the compounds in an assay to measure non-homologous end joining editing efficiency in primary T cells at 30° C.

FIG. 3C shows a dose response curve of the compounds in an assay to measure primary T cell yield at 37° C.

FIG. 3D shows a dose response curve of the compounds in an assay to measure primary T cell yield at 30° C.

FIG. 4 shows concentration-dependent increase in the frequency of HDR events (% GFP+ cells) in T cells from multiple donors cultured with aminacrine following megaTAL.

FIG. 5 shows that aminacrine induced a concentration-dependent increase in HDR frequency in CD34+ cells at the targeted BCL11A locus, but not at the non-target CCR5 locus.

FIG. 6 shows the results from a lineage analysis for methylcellulose cultured CD34+ cells treated with megaTAL alone, megaTAL with rAAV, with or without aminacrine.

FIG. 7 shows elevated HDR rates in methylcellulose colonies derived from primary CD34+ cells treated with megaTAL alone, megaTAL with rAAV, with or without aminacrine.

FIG. 8 shows that aminacrine increases HDR in bulk human CD34⁺ cells electroporated with a BCL11A targeting megaTAL and transduced with an AAV donor repair template compared to cells that were not treated with aminacrine.

### BRIEF DESCRIPTION OF THE SEQUENCE IDENTIFIERS

SEQ ID NOs: 1-11 set forth the amino acid sequences of various linkers.

SEQ ID NOs: 12-14 set forth the amino acid sequences of protease cleavage sites and self-cleaving polypeptide cleavage sites.

### DETAILED DESCRIPTION

### A. Overview

Gene therapies may rely, in part, on genome editing to obtain sufficient therapeutic gene expression and/or to eliminate expression of genes that negatively influence or reduce the efficacy of the gene therapy. One of the main limitations of implementing a genome editing strategy is the low efficiency of genome editing. The genome editing strategies contemplated herein, and methods of making and using the same to generate improved gene therapies, solve these and other problems plaguing the art.

Various embodiments contemplated herein, generally relate to, in part, improved genome editing compositions. The genome editing compositions represent a quantum improvement in generating gene therapies for the treatment of monogenetic disorders, diseases, and conditions, e.g., hemoglobinopathies, cancer, infectious disease, autoimmune disease, inflammatory disease, and immunodeficiency. Gene therapies manufactured using the genome editing compositions and methods contemplated herein offer numerous advantages compared to existing gene therapies including, but not limited to, decreased cost of goods to generate the therapeutics, expanded range of gene therapies to cells with historically low genome editing efficiencies, and increased potency of gene therapy compositions.

Genome editing compositions and methods contemplated in particular embodiments comprise genome editing enhancers that increase the rate of homology directed repair (HDR) and non-homologous end joining (NHEJ) in nuclease-based gene editing strategies used to manufacture gene therapies.

Various embodiments contemplate genome editing compositions comprising a genome editing enhancer and an engineered nuclease. In particular embodiments, the genome editing enhancer is preferably a nucleic acid intercalator, more preferably the genome editing enhancer is a DNA intercalator, even more preferably the genome editing enhancer is an acridine, and even more preferably the genome editing enhancer is 9-aminoacridine.

Various other embodiments contemplate methods to increase genome editing efficiency comprising introducing an engineered nuclease and a nucleic acid intercalator into a population of cells, in amounts and for a time sufficient to increase the frequency of genome editing in the cells, compared to cells where a nucleic acid intercalator has not been introduced.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism, such as cell self-renewal and cell proliferation or expansion. In one embodiment, the term “in vivo expansion” refers to the ability of a cell population to increase in number in vivo. In one embodiment, cells are engineered or modified in vivo.

As used herein, the term “amount” refers to “an amount effective” or “an effective amount” of a compound, composition, or treatment sufficient to achieve a desired result, e.g., a desired rate of genome editing in a population of cells.

By “enhance” or “promote” or “increase” or “expand” or “potentiate” refers generally to the ability of a composition contemplated herein to produce, elicit, or cause a greater response (i.e., physiological response) compared to the response caused by either vehicle or a control molecule/composition. A measurable response may include an increase in HR or HDR efficiency. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower” or “lessen” or “reduce” or “abate” or “ablate” or “inhibit” or “dampen” refers generally to the ability of composition contemplated herein to produce, elicit, or cause a lesser response (i.e., physiological response) compared to the response caused by either vehicle or a control molecule/composition. A measurable response may include a decrease in endogenous gene expression or function, a decrease in expression of biomarkers associated with immune effector cell exhaustion, and the like. A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a substantially similar or comparable physiological response (i.e., downstream effects) in a cell, as compared to the response caused by either vehicle, a control molecule/composition, or the response in a particular cell lineage. A comparable response is one that is not significantly different or measurable different from the reference response.

A “small molecule,” “small organic molecule,” or “small molecule compound” refers to a low molecular weight compound that has a molecular weight of less than about 5 kD, less than about 4 kD, less than about 3 kD, less than about 2 kD, less than about 1 kD, or less than about 0.5 kD. In particular embodiments, small molecules can include, nucleic acids, peptides, peptidomimetics, peptoids, other small organic compounds or drugs, and the like. Libraries of chemical and/or biological mixtures, such as fungal, bacterial, or algal extracts, are known in the art and can be screened with any of the assays of the invention. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair (HDR) mechanisms. This process requires nucleotide sequence homology, uses a “donor molecule” or “donor repair template” as a template to repair a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“NHEJ” or “non-homologous end joining” refers to the resolution of a double-strand break in the absence of a donor repair template or homologous sequence. NHEJ can result in insertions and deletions at the site of the break. NHEJ is mediated by several sub-pathways, each of which has distinct mutational consequences. The classical NHEJ pathway (cNHEJ) requires the KU/DNA-PKcs/Lig4/XRCC4 complex, ligates ends back together with minimal processing and often leads to precise repair of the break. Alternative NHEJ pathways (altNHEJ) also are active in resolving dsDNA breaks, but these pathways are considerably more mutagenic and often result in imprecise repair of the break marked by insertions and deletions. While not wishing to be bound to any particular theory, it is contemplated that modification of dsDNA breaks by end-processing enzymes, such as, for example, exonucleases, e.g., Trex2, may bias repair towards an altNHEJ pathway.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, polypeptides contemplated herein are used for targeted double-stranded DNA cleavage.

A “target site” or “target sequence” is a chromosomal or extrachromosomal nucleic acid sequence that defines a portion of a nucleic acid to which a binding molecule will bind and/or cleave, provided sufficient conditions for binding and/or cleavage exist.

An “exogenous” molecule is a molecule that is not normally present in a cell, but that is introduced into a cell by one or more genetic, biochemical or other methods. Exemplary exogenous molecules include, but are not limited to small organic molecules, e.g., DNA intercalators, protein, nucleic acid, carbohydrate, lipid, glycoprotein, lipoprotein, polysaccharide, any modified derivative of the above molecules, or any complex comprising one or more of the above molecules. Illustrative methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. For example, an endogenous nucleic acid can comprise a chromosome, the genome of a mitochondrion, or other organelle, or a naturally-occurring episomal nucleic acid.

A “gene,” refers to a DNA region encoding a gene product, as well as all DNA regions which regulate the production of the gene product, whether or not such regulatory sequences are adjacent to coding and/or transcribed sequences. A gene includes, but is not limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “genome editing” refers to the substitution, deletion, and/or introduction of genetic material at a target site in the cell's genome, which restores, corrects, and/or modifies expression of a gene, and/or for the purpose of expressing one or more immunopotency enhancers, immunosuppressive signal dampers, and engineered antigen receptors. Genome editing contemplated in particular embodiments comprises introducing a genome editing enhancer and one or more engineered nucleases (or mRNA encoding the same) into a cell to generate DNA lesions at a target site in the cell's genome, optionally in the presence of a donor repair template.

As used herein, the term “genetically engineered” or “genetically modified” refers to the chromosomal or extrachromosomal addition of extra genetic material in the form of DNA or RNA to the total genetic material in a cell. Genetic modifications may be targeted or non-targeted to a particular site in a cell's genome. In one embodiment, genetic modification is site specific. In one embodiment, genetic modification is not site specific.

### C. Genome Editing Enhancers

One limitation of using genome editing strategies is the low efficiency. The genome editing compositions and methods contemplated in particular embodiments solve the problem of inefficient editing by using a genome editing enhancer. As used herein, the term “genome editing enhancer” refers to a small molecule or compound that increases homology directed repair (HDR) and/or error prone non-homologous end joining (NHEJ). Genome editing enhancers suitable for use in compositions and methods contemplated in particular embodiments include, but are not limited to nucleic acid intercalating agents.

As used herein, the terms “intercalating agent” or “intercalator” are known in the art to refer to those compounds capable of non-covalent insertion between the base pairs of a nucleic acid duplex and are specific in this regard only to double-stranded (ds) portions of nucleic acid structures including those portions of single-stranded nucleic acids which have formed base pairs, such as in “hairpin loops”. The nucleic acid structures can be dsDNA, dsRNA or DNA-RNA hybrids. The term “intercalating agent or intercalator” is also used to describe the insertion of planar aromatic or heteroaromatic compounds between adjacent base pairs of double stranded DNA (dsDNA), or in some cases dsRNA. In particular embodiments, the efficiency of genome editing is preferably increased using a genome editing enhancer, more preferably using a nucleic acid intercalator, more preferably a DNA intercalator, even more preferably an acridine, and even more preferably 9-aminoacridine.

Illustrative examples of genome editing enhancers that are suitable for use in particular compositions and methods contemplated herein include, but are not limited to monofunctional intercalating agents, bifunctional intercalating agents, and polyfunctional intercalating agents.

Additional illustrative examples of genome editing enhancers that are suitable for use in particular compositions and methods contemplated herein include, but are not limited to acridines, anthracyclines, alkaloids, coumarins, phenanthridines, and naphthalimides.

In particular illustrative embodiments, the genome editing enhancer is selected from the group consisting of: 1,8-naphthalimide, 4′6-diamidino-α-phenylindole, acridines, acridine orange, acriflavine, acronycine, actinodaphnidine, aminacrine, amsacrine, anthracycline, anthramycin, anthrapyrazole, benzophenanthridine alkaloids, berbamine, berberine, berberrubine, bleomycin, BOBO-1, BOBO-3, boldine, BO-PRO-1, BO-PRO-3, bublocapnine, camptothecin, cassythine, chartreusin, chloroquine, chromomycin, cinchonidine, cinchonine, coptisine, coralyne, coumarin, cryptolepine, dactinomycin, DAPI, daunorubicin, dicentrine, dictamine, distamycin, doxorubicin, ellipticine, emetine, ethacridine, ethidium, evolitrine, fagarine, fagaronine, fluorcoumanin, GelStar, gentamicin, glaucine, harmaline, harmine, harmine, hedamycin, hexidium, Hoechst 33258, Hoechst 33342, homidium, hycanthone, imidazoacridinone, an indazole analog, iodide, isocorydine, isoquinoline alkaloids, jatrorrhizine, JOJO-1, JO-PRO-1, kinetin riboside, kokusainine, lobeline, LOLO-1, LO-PRO-1, lucanthone, masculine, matadine, mepacrine, a metallo-intercalator, mithramycin, mitoxantrone, neocryptolepine, netropsin, nitidine, nitracrine, nogalamycin, norharman, OliGreen, palmatine, phenanthridine, PicoGreen, pirarubicin, polypyridyls, POPO-1, POPO-3, PO-PRO-1, PO-PRO-3, proflavine, propidium, psoralen, quinacrine, quinidine, quinine, quinoxalines, RiboGreen, a rhodium based intercalator, a ruthenium based intercalator, sanguinarine, serpentine, skimmianine, streptomycin, SYBR DX, SYBR Gold, SYBR Green I, SYBR Green II, SYTO-11, SYTO-12, SYTO-13, SYTO-14, SYTO-15, SYTO-16, SYTO-17, SYTO-20, SYTO-21, SYTO-22, SYTO-23, SYTO-24, SYTO-25, SYTO-40, SYTO-41, SYTO-42, SYTO-43, SYTO-44, SYTO-45, SYTO-59, SYTO-60, SYTO-61, SYTO-62, SYTO-63, SYTO-64, SYTO-80, SYTO-81, SYTO-82, SYTO-83, SYTO-84, SYTO-85, SYTOX blue, SYTOX green, SYTOX orange, tacrine, thalidomide, thiazole orange, tilorone, TO-PRO-1, TO-PRO-3, TO-PRO-5, TOTO-1, TOTO-3, usambarensine, YO-PRO-1, YO-PRO-3, YOYO-1, YOYO-3, and analogs and derivatives thereof.

In a particular embodiment, the genome editing enhancer is selected from the group consisting of: tilorone, aminacrine, homidium bromide (ethidium bromide), harmine, hycanthone, daunorubicin, sanguinarine sulfate, kinetin riboside, ethacridine lactate, and cyclohexamide.

In a particular embodiment, the genome editing enhancer is selected from the group consisting of: tilorone, aminacrine, homidium bromide (ethidium bromide), and harmine.

In a preferred embodiment, the genome editing enhancer is an acridine or diacridine.

In another preferred embodiment, the genome editing enhancer is aminacrine (9-aminoacridine).

### D. Nucleases

Engineered nucleases targeting one or more target sites in a cell are used in the genome editing compositions and methods contemplated herein. An “engineered nuclease” refers to a nuclease comprising one or more DNA binding domains and one or more DNA cleavage domains, wherein the nuclease has been designed and/or modified to bind a DNA binding target sequence adjacent to a DNA cleavage target sequence. The engineered nuclease may be designed and/or modified from a naturally occurring nuclease or from a previously engineered nuclease. Engineered nucleases contemplated in particular embodiments may further comprise one or more additional functional domains, e.g., an end-processing enzymatic domain of an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity.

The engineered nucleases contemplated in particular embodiments generate single-stranded DNA nicks or double-stranded DNA breaks (DSB) in a target sequence. Furthermore, a DSB can be achieved in the target DNA by the use of two nucleases generating single-stranded nicks (nickases). Each nickase cleaves one strand of the DNA and the use of two or more nickases can create a double strand break (e.g., a staggered double-stranded break) in a target DNA sequence. In particular embodiments, the nucleases are used in combination with a donor repair template, which is introduced into the target sequence at the DNA break-site via homologous recombination at a DSB.

Illustrative examples of nucleases that may be engineered to bind and cleave a target sequence include, but are not limited to homing endonucleases (meganucleases), megaTALs, transcription activator-like effector nucleases (TALENs), zinc finger nucleases (ZFNs), ARCUS nucleases, and clustered regularly-interspaced short palindromic repeats (CRISPR)/Cas nuclease systems.

In various embodiments, a homing endonuclease or meganuclease is engineered to bind to, and to introduce single-stranded nicks or double-strand breaks (DSBs) in, one or more target sites in a cell. “Homing endonuclease” and “meganuclease” are used interchangeably and refer to naturally-occurring nucleases or engineered meganucleases that recognize 12-45 base-pair cleavage sites and are commonly grouped into five families based on sequence and structure motifs: LAGLIDADG, GIY-YIG, HNH, His-Cys box, and PD-(D/E)XK.

A “reference homing endonuclease” or “reference meganuclease” refers to a wild type homing endonuclease or a homing endonuclease found in nature. In one embodiment, a “reference homing endonuclease” refers to a wild type homing endonuclease that has been modified to increase basal activity.

An “engineered homing endonuclease,” “reprogrammed homing endonuclease,” “homing endonuclease variant,” “engineered meganuclease,” “reprogrammed meganuclease,” or “meganuclease variant” refers to a homing endonuclease comprising one or more DNA binding domains and one or more DNA cleavage domains, wherein the homing endonuclease has been designed and/or modified from a parental or naturally occurring homing endonuclease, to bind and cleave a DNA target sequence. The homing endonuclease variant may be designed and/or modified from a naturally occurring homing endonuclease or from another homing endonuclease variant. Homing endonuclease variants contemplated in particular embodiments may further comprise one or more additional functional domains, e.g., an end-processing enzymatic domain of an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity.

Homing endonuclease (HE) variants do not exist in nature and can be obtained by recombinant DNA technology or by random mutagenesis. HE variants may be obtained by making one or more amino acid alterations, e.g., mutating, substituting, adding, or deleting one or more amino acids, in a naturally occurring HE or HE variant. In particular embodiments, a HE variant comprises one or more amino acid alterations to the DNA recognition interface.

HE variants contemplated in particular embodiments may further comprise one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerase activity. In particular embodiments, HE variants are introduced into a T cell with an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerase activity. The HE variant and 3′ processing enzyme may be introduced separately, e.g., in different vectors or separate mRNAs, or together, e.g., as a fusion protein, or in a polycistronic construct separated by a viral self-cleaving peptide or an IRES element.

Illustrative examples of LAGLIDADG homing endonucleases (LHE) from which reprogrammed LHEs or LHE variants may be designed include, but are not limited to: I-CreI and I-SceI.

Additional illustrative examples of LAGLIDADG homing endonucleases (LHE) from which reprogrammed LHEs or LHE variants may be designed include, but are not limited to: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

In one embodiment, the reprogrammed LHEs or LHE variants are selected from the group consisting of: I-CpaMI, I-HjeMI, I-OnuI, I-PanMI, and SmaMI.

In one embodiment, the reprogrammed LHE or LHE variant is I-OnuI.

In one embodiment, reprogrammed LHEs or LHE variants are generated from a natural I-OnuI. In a preferred embodiment, reprogrammed LHEs or LHE variants are generated from a previously engineered I-OnuI.

In a particular embodiment, reprogrammed LHEs or LHE variants comprises one or more amino acid substitutions in the DNA recognition interface. In particular embodiments, the I-OnuI LHE comprises at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the DNA recognition interface of I-OnuI (Taekuchi et al. 2011. Proc Natl Acad Sci U.S.A. 2011 Aug. 9; 108(32): 13077-13082) or an engineered variant of I-OnuI.

In one embodiment, reprogrammed LHEs or LHE variants comprise at least 70%, more preferably at least 80%, more preferably at least 85%, more preferably at least 90%, more preferably at least 95%, more preferably at least 97%, more preferably at least 99% sequence identity with the DNA recognition interface of I-OnuI (Taekuchi et al. 2011. Proc Natl Acad Sci U.S.A. 2011 Aug. 9; 108(32): 13077-13082) or an engineered variant of I-OnuI.

Various illustrative embodiments contemplate a megaTAL nuclease that binds to and cleaves a target region of one or more target sites. A “megaTAL” refers to an engineered nuclease comprising an engineered TALE DNA binding domain and an engineered meganuclease, and optionally comprise one or more linkers and/or additional functional domains, e.g., an end-processing enzymatic domain of an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity. In particular embodiments, a megaTAL can be introduced into a T cell with an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity. The megaTAL and 3′ processing enzyme may be introduced separately, e.g., in different vectors or separate mRNAs, or together, e.g., as a fusion protein, or in a polycistronic construct separated by a viral self-cleaving peptide or an IRES element.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, e.g., AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brg11 and hpx17 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

In particular embodiments, a megaTAL comprises a TALE DNA binding domain comprising one or more repeat units that are involved in binding of the TALE DNA binding domain to its corresponding target DNA sequence. A single “repeat unit” (also referred to as a “repeat”) is typically 33-35 amino acids in length. Each TALE DNA binding domain repeat unit includes 1 or 2 DNA-binding residues making up the Repeat Variable Di-Residue (RVD), typically at positions 12 and/or 13 of the repeat. The natural (canonical) code for DNA recognition of these TALE DNA binding domains has been determined such that an HD sequence at positions 12 and 13 leads to a binding to cytosine (C), NG binds to T, NI to A, NN binds to G or A, and NG binds to T. In certain embodiments, non-canonical (atypical) RVDs are contemplated.

Illustrative examples of non-canonical RVDs suitable for use in particular megaTALs contemplated in particular embodiments include, but are not limited to HH, KH, NH, NK, NQ, RH, RN, SS, NN, SN, KN for recognition of guanine (G); NI, KI, RI, HI, SI for recognition of adenine (A); NG, HG, KG, RG for recognition of thymine (T); RD, SD, HD, ND, KD, YG for recognition of cytosine (C); NV, HN for recognition of A or G; and H*, HA, KA, N*, NA, NC, NS, RA, S*for recognition of A or T or G or C, wherein (*) means that the amino acid at position 13 is absent. Additional illustrative examples of RVDs suitable for use in particular megaTALs contemplated in particular embodiments further include those disclosed in U.S. Pat. No. 8,614,092, which is incorporated herein by reference in its entirety.

In particular embodiments, a megaTAL contemplated herein comprises a TALE DNA binding domain comprising 3 to 30 repeat units. In certain embodiments, a megaTAL comprises 3, 4, 5, 6, 7, 8, 9, 10, 11, 12, 13, 14, 15, 16, 17, 18, 19, 20, 21, 22, 23, 24, 25, 26, 27, 28, 29, or 30 TALE DNA binding domain repeat units. In a preferred embodiment, a megaTAL contemplated herein comprises a TALE DNA binding domain comprising 5-13 repeat units, more preferably 7-12 repeat units, more preferably 9-11 repeat units, and more preferably 9, 10, or 11 repeat units.

In particular embodiments, a megaTAL contemplated herein comprises a TALE DNA binding domain comprising 3 to 30 repeat units and an additional single truncated TALE repeat unit comprising 20 amino acids located at the C-terminus of a set of TALE repeat units, i.e., an additional C-terminal half-TALE DNA binding domain repeat unit (amino acids −20 to −1 of the C-cap disclosed elsewhere herein, infra). Thus, in particular embodiments, a megaTAL contemplated herein comprises a TALE DNA binding domain comprising 3.5 to 30.5 repeat units. In certain embodiments, a megaTAL comprises 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 TALE DNA binding domain repeat units. In a preferred embodiment, a megaTAL contemplated herein comprises a TALE DNA binding domain comprising 5.5-13.5 repeat units, more preferably 7.5-12.5 repeat units, more preferably 9.5-11.5 repeat units, and more preferably 9.5, 10.5, or 11.5 repeat units.

In particular embodiments, a megaTAL comprises an “N-terminal domain (NTD)” polypeptide, one or more TALE repeat domains/units, a “C-terminal domain (CTD)” polypeptide, an engineered meganuclease, and one or more linker peptides joining the domains. As used herein, the term “N-terminal domain (NTD)” polypeptide refers to the sequence that flanks the N-terminal portion or fragment of a naturally occurring TALE DNA binding domain. As used herein, the term “C-terminal domain (CTD)” polypeptide refers to the sequence that flanks the C-terminal portion or fragment of a naturally occurring TALE DNA binding domain.

In particular embodiments, a megaTAL contemplated herein, comprises an NTD of about 122 amino acids to 137 amino acids, about 9.5, about 10.5, or about 11.5 binding repeat units, a CTD of about 20 amino acids to about 85 amino acids, and an engineered I-OnuI LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I, or preferably I-CpaMI, I-HjeMI, I-OnuI, I-PanMI, and SmaMI, or more preferably I-OnuI.

In particular embodiments, the engineered nuclease is a TALEN. A “TALEN” refers to an engineered nuclease comprising an engineered TALE DNA binding domain and an endonuclease domain (or endonuclease half-domain thereof), and optionally comprise one or more linkers and/or additional functional domains, e.g., an end-processing enzymatic domain of an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity. In particular embodiments, a TALEN can be introduced into a T cell with an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity. The TALEN and 3′ processing enzyme may be introduced separately, e.g., in different vectors or separate mRNAs, or together, e.g., as a fusion protein, or in a polycistronic construct separated by a viral self-cleaving peptide or an IRES element.

TALENs contemplated in particular embodiments comprise an NTD, a TALE DNA binding domain comprising about 3.5 to 30.5 repeat units, e.g., about 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 repeat units, a CTD, and an endonuclease domain or half-domain.

In one embodiment, a TALEN contemplated herein comprises an endonuclease domain of a Type-IIS restriction endonuclease. In one embodiment, the Type-IIS restriction endonuclease is Fok I.

Illustrative examples of TALENs and methods of making the same are disclosed in U.S. Pat. Nos. 8,586,526; 8,912,138; and 9,315,788, each of which is incorporated herein by reference in its entirety.

In particular embodiments, the engineered nuclease is a zinc finger nuclease (ZFN). A “ZFN” refers to an engineered nuclease comprising one or more zinc finger DNA binding domains and an endonuclease domain (or endonuclease half-domain thereof), and optionally comprise one or more linkers and/or additional functional domains, e.g., an end-processing enzymatic domain of an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity. In particular embodiments, a ZFN can be introduced into a T cell with an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity. The ZFN and 3′ processing enzyme may be introduced separately, e.g., in different vectors or separate mRNAs, or together, e.g., as a fusion protein, or in a polycistronic construct separated by a viral self-cleaving peptide or an IRES element.

In particular embodiments, the ZFN comprises a zinger finger DNA binding domain that has one, two, three, four, five, six, seven, or eight or more zinger finger motifs and an endonuclease domain (or endonuclease half-domain). Typically, a single zinc finger motif is about 30 amino acids in length. Zinc fingers motifs include both canonical C₂H₂ zinc fingers, and non-canonical zinc fingers such as, for example, C₃H zinc fingers and C₄ zinc fingers.

Zinc finger binding domains can be engineered to bind any DNA sequence. Individual zinc finger motifs bind to a three or four nucleotide sequence. Candidate zinc finger DNA binding domains for a given 3 bp DNA target sequence have been identified and modular assembly strategies have been devised for linking a plurality of the domains into a multi-finger peptide targeted to the corresponding composite DNA target sequence. Other suitable methods known in the art can also be used to design and construct nucleic acids encoding zinc finger DNA binding domains, e.g., phage display, random mutagenesis, combinatorial libraries, computer/rational design, affinity selection, PCR, cloning from cDNA or genomic libraries, synthetic construction and the like. (See, e.g., U.S. Pat. No. 5,786,538; Wu et al., PNAS 92:344-348 (1995); Jamieson et al., Biochemistry 33:5689-5695 (1994); Rebar & Pabo, Science 263:671-673 (1994); Choo & Klug, PNAS 91:11163-11167 (1994); Choo & Klug, PNAS 91: 11168-11172 (1994); Desjarlais & Berg, PNAS 90:2256-2260 (1993); Desjarlais & Berg, PNAS 89:7345-7349 (1992); Pomerantz et al., Science 267:93-96 (1995); Pomerantz et al., PNAS 92:9752-9756 (1995); Liu et al., PNAS 94:5525-5530 (1997); Griesman & Pabo, Science 275:657-661 (1997); Desjarlais & Berg, PNAS 91:11-99-11103 (1994)).

In particular embodiments, ZNFs contemplated herein comprise, a zinc finger DNA binding domain comprising two, three, four, five, six, seven or eight or more zinc finger motifs, and an endonuclease domain or half-domain from at least one Type-IIS restriction enzyme. In one embodiment, the endonuclease domain or half-domain is from the Fok I Type-IIS restriction endonuclease.

Illustrative examples of ZNFs and methods of making the same are disclosed in U.S. Patent Publication Nos.: 20030232410; 20050208489; 20050026157; 20050064474; 20060188987; 20060063231, each of which is incorporated herein by reference in its entirety.

In various embodiments, a CRISPR (Clustered Regularly Interspaced Short Palindromic Repeats)/Cas (CRISPR Associated) nuclease system is engineered to bind to, and to introduce single-stranded nicks or double-strand breaks (DSBs) in, one or more target sites. The CRISPR/Cas nuclease system is a recently engineered nuclease system based on a bacterial system that can be used for mammalian genome engineering. See, e.g., Jinek et al. (2012) Science 337:816-821; Cong et al. (2013) Science 339:819-823; Mali et al. (2013) Science 339:823-826; Qi et al. (2013) Cell 152:1173-1183; Jinek et al. (2013), eLife 2:e00471; David Segal (2013) eLife 2:e00563; Ran et al. (2013) Nature Protocols 8(11):2281-2308; Zetsche et al. (2015) Cell 163(3):759-771, each of which is incorporated herein by reference in its entirety.

In one embodiment, the CRISPR/Cas nuclease system comprises Cas nuclease and one or more RNAs that recruit the Cas nuclease to the target site, e.g., a transactivating cRNA (tracrRNA) and a CRISPR RNA (crRNA), or a single guide RNA (sgRNA). crRNA and tracrRNA can engineered into one polynucleotide sequence referred to herein as a “single guide RNA” or “sgRNA.”

In one embodiment, the Cas nuclease is engineered as a double-stranded DNA endonuclease or a nickase or catalytically dead Cas, and forms a target complex with a crRNA and a tracrRNA, or sgRNA, for site specific DNA recognition and site-specific cleavage of the protospacer target sequence located within the target site. The protospacer motif abuts a short protospacer adjacent motif (PAM), which plays a role in recruiting a Cas/RNA complex. Cas polypeptides recognize PAM motifs specific to the Cas polypeptide. Accordingly, the CRISPR/Cas system can be used to target and cleave either or both strands of a double-stranded polynucleotide sequence flanked by particular 3′ PAM sequences specific to a particular Cas polypeptide. PAMs may be identified using bioinformatics or using experimental approaches. Esvelt et al., 2013, Nature Methods. 10(11):1116-1121, which is hereby incorporated by reference in its entirety.

In various embodiments, the Cas nuclease is Cas9 or Cpf1.

Illustrative examples of Cas9 polypeptides suitable for use in particular embodiments contemplated in particular embodiments may be obtained from bacterial species including, but not limited to: Enterococcus faecium, Enterococcus italicus, Listeria innocua, Listeria monocytogenes, Listeria seeligeri, Listeria ivanovii, Streptococcus agalactiae, Streptococcus anginosus, Streptococcus bovis, Streptococcus dysgalactiae, Streptococcus equinus, Streptococcus gallolyticus, Streptococcus macacae, Streptococcus mutans, Streptococcus pseudoporcinus, Streptococcus pyogenes, Streptococcus thermophilus, Streptococcus gordonii, Streptococcus infantarius, Streptococcus macedonicus, Streptococcus mitis, Streptococcus pasteurianus, Streptococcus suis, Streptococcus vestibularis, Streptococcus sanguinis, Streptococcus downei, Neisseria bacilliformis, Neisseria cinerea, Neisseria flavescens, Neisseria lactamica, Neisseria meningitidis, Neisseria subflava, Lactobacillus brevis, Lactobacillus buchneri, Lactobacillus casei, Lactobacillus paracasei, Lactobacillus fermentum, Lactobacillus gasseri, Lactobacillus jensenii, Lactobacillus johnsonii, Lactobacillus rhamnosus, Lactobacillus ruminis, Lactobacillus salivarius, Lactobacillus sanfranciscensis, Corynebacterium accolens, Corynebacterium diphtheriae, Corynebacterium matruchotii, Campylobacter jejuni, Clostridium perfringens, Treponema vincentii, Treponema phagedenis, and Treponema denticola.

Illustrative examples of Cpf1 polypeptides suitable for use in particular embodiments contemplated in particular embodiments may be obtained from bacterial species including, but not limited to: Francisella spp., Acidaminococcus spp., Prevotella spp., Lachnospiraceae spp., among others.

Conserved regions of Cas9 orthologs include a central HNH endonuclease domain and a split RuvC/RNase H domain. Cpf1 orthologs possess a RuvC/RNase H domain but no discernable HNH domain. The HNH and RuvC-like domains are each responsible for cleaving one strand of the double-stranded DNA target sequence. The HNH domain of the Cas9 nuclease polypeptide cleaves the DNA strand complementary to the tracrRNA:crRNA or sgRNA. The RuvC-like domain of the Cas9 nuclease cleaves the DNA strand that is not-complementary to the tracrRNA:crRNA or sgRNA. Cpf1 is predicted to act as a dimer wherein each RuvC-like domain of Cpf1 cleaves either the complementary or non-complementary strand of the target site. In particular embodiments, a Cas9 nuclease variant (e.g., Cas9 nickase) is contemplated comprising one or more amino acids additions, deletions, mutations, or substitutions in the HNH or RuvC-like endonuclease domains that decreases or eliminates the nuclease activity of the variant domain.

Illustrative examples of Cas9 HNH mutations that decrease or eliminate the nuclease activity in the domain include, but are not limited to: S. pyogenes (D10A); S. thermophilis (D9A); T. denticola (D13A); and N. meningitidis (D16A).

Illustrative examples of Cas9 RuvC-like domain mutations that decrease or eliminate the nuclease activity in the domain include, but are not limited to: S. pyogenes (D839A, H840A, or N863A); S. thermophilis (D598A, H599A, or N622A); T. denticola (D878A, H879A, or N902A); and N. meningitidis (D587A, H588A, or N611A).

### E. Target Sites

Engineered nucleases contemplated in particular embodiments can be designed to bind to any suitable target sequence and can have a novel binding specificity, compared to a naturally-occurring nuclease. In particular embodiments, the target site is a regulatory region of a gene including, but not limited to promoters, enhancers, repressor elements, and the like. In particular embodiments, the target site is a coding region of a gene or a splice site. In certain embodiments, engineered nucleases are designed to down-regulate or decrease expression of a gene. An engineered nuclease and donor repair template can be designed to delete a desired target sequence. In some embodiments, engineered nucleases and donor repair templates are designed to correct a mutation in the coding sequence of a gene or regulatory region and/or to restore normal function to the polypeptide encoded by the gene or its regulatory region.

Illustrative examples of suitable target sequences include the following genes: 13 globin, δ globin, γ globin, B-cell lymphoma/leukemia 11(BCL11A), Kruppel-like factor 1 (KLF1), CCR5, CXCR4, PPP1R12C (AAVS1), hypoxanthine phosphoribosyltransferase (HPRT), albumin, Factor VIII, Factor IX, Leucine-rich repeat kinase 2 (LRRK2), Hungtingin (Htt), superoxide dismutase 1 (SOD1), C9orf72, TARDBP, FUS, rhodopsin (RHO), Cystic Fibrosis Transmembrane Conductance Regulator (CFTR), surfactant protein B (SFTPB), T cell receptor alpha (TRAC), T cell receptor beta (TRBC), programmed cell death 1 (PD1), Cytotoxic T-Lymphocyte Antigen 4 (CTLA-4), human leukocyte antigen (HLA) A, HLA B, HLA C, HLA-DP, HLA-DQ, HLA-DR, LMP7, Transporter associated with Antigen Processing (TAP) 1, TAP2, tapasin (TAPBP), class II major histocompatibility complex transactivator (CIITA), dystrophin (DMD), glucocorticoid receptor (GR), IL2RG, Rag-1, RFX5, FAD2, FAD3, ZP15, KASII, MDH, and EPSPS.

Additional illustrative examples of suitable target sites for insertion of donor templates encoding therapeutic transgenes include, but are not limited to “safe harbor” loci such as the AAVS1, HPRT, albumin, and CCR5 genes.

### F. Donor Repair Templates

Cell-based compositions contemplated in particular embodiments are generated by genome editing with engineered nucleases, genome editing enhancers, and introduction of one or more donor repair templates. Without wishing to be bound by any particular theory, it is contemplated that expression of one or more engineered nucleases in a cell generates single- or double-stranded DNA breaks at a target site; and that nuclease expression and break generation in the presence of a genome editing enhancer and a donor repair template leads to insertion or integration of the template at the target site by homologous recombination, thereby repairing the break.

In particular embodiments, the donor repair template comprises one or more homology arms.

In particular embodiments, the donor repair template comprises one or more homology arms that flank the DSB site.

As used herein, the term “homology arms” refers to a nucleic acid sequence in a donor repair template that is identical, or nearly identical, to DNA sequence flanking the DNA break introduced by the nuclease at a target site. In one embodiment, the donor repair template comprises a 5′ homology arm that comprises a nucleic acid sequence that is identical or nearly identical to the DNA sequence 5′ of the DNA break site. In one embodiment, the donor repair template comprises a 3′ homology arm that comprises a nucleic acid sequence that is identical or nearly identical to the DNA sequence 3′ of the DNA break site. In a preferred embodiment, the donor repair template comprises a 5′ homology arm and a 3′ homology arm. The donor repair template may comprise homology to the genome sequence immediately adjacent to the DSB site, or homology to the genomic sequence within any number of base pairs from the DSB site. In one embodiment, the donor repair template comprises a nucleic acid sequence that is homologous to a genomic sequence about 5 bp, about 10 bp, about 25 bp, about 50 bp, about 100 bp, about 250 bp, about 500 bp, about 1000 bp, about 2500 bp, about 5000 bp, about 10000 bp or more, including any intervening length of homologous sequence.

Illustrative examples of suitable lengths of homology arms contemplated in particular embodiments, may be independently selected, and include but are not limited to: about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, about 1600 bp, about 1700 bp, about 1800 bp, about 1900 bp, about 2000 bp, about 2100 bp, about 2200 bp, about 2300 bp, about 2400 bp, about 2500 bp, about 2600 bp, about 2700 bp, about 2800 bp, about 2900 bp, or about 3000 bp, or longer homology arms, including all intervening lengths of homology arms.

Additional illustrative examples of suitable homology arm lengths include, but are not limited to: about 100 bp to about 3000 bp, about 200 bp to about 3000 bp, about 300 bp to about 3000 bp, about 400 bp to about 3000 bp, about 500 bp to about 3000 bp, about 500 bp to about 2500 bp, about 500 bp to about 2000 bp, about 750 bp to about 2000 bp, about 750 bp to about 1500 bp, or about 1000 bp to about 1500 bp, including all intervening lengths of homology arms.

In a particular embodiment, the lengths of the 5′ and 3′ homology arms are independently selected from about 500 bp to about 1500 bp. In one embodiment, the 5′homology arm is about 1500 bp and the 3′ homology arm is about 1000 bp. In one embodiment, the 5′homology arm is about 600 bp and the 3′ homology arm is about 600 bp.

Donor repair templates may further comprises one or more polynucleotides such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, contemplated elsewhere herein.

In various embodiments, the donor repair template comprises a 5′ homology arm, an RNA polymerase II promoter, one or more polynucleotides encoding a therapeutic gene or fragment thereof, transgene or selectable marker, and a 3′ homology arm.

In various embodiments, a target site is modified with a donor repair template comprising a 5′ homology arm, one or more polynucleotides encoding a therapeutic gene or fragment thereof, transgene or selectable marker, and a 3′ homology arm.

In various embodiments, the donor repair template comprises one or more polynucleotides encoding a therapeutic gene or fragment thereof, transgene, or selectable marker.

In various embodiments, the donor repair template comprises one or more polynucleotides encoding a therapeutic gene or fragment thereof, transgene, or selectable marker including, but not limited to: β globin, δ globin, γ globin, BCL11A, KLF1, CCR5, CXCR4, PPP1R12C (AAVS1), HPRT, albumin, Factor VIII, Factor IX, LRRK2, Htt, SOD1, C9orf72, TARDBP, FUS, RHO, CFTR, SFTPB, TRAC, TRBC, PD1, CTLA-4, HLA A, HLA B, HLA C, HLA-DP, HLA-DQ, HLA-DR, LMP7, TAP 1, TAP2, TAPBP, CIITA, DMD, GR, IL2RG, Rag-1, RFX5, FAD2, FAD3, ZP15, KASII, MDH, and EPSPS.

In various embodiments, the donor repair template comprises one or more polynucleotides encoding a therapeutic gene or fragment thereof selected from the group consisting of: cytokines, lymphokines, monokines, chemokines, hormones, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, fibroblast growth factor, prolactin, placental lactogen, mullerian-inhibiting substance, mouse gonadotropin-associated peptide, inhibin, activing, vascular endothelial growth factor, integrin, thrombopoietin (TPO), nerve growth factors such as NGF-beta, platelet-growth factor, transforming growth factors (TGFs) such as TGF-alpha and TGF-beta; insulin-like growth factor-I and -II, erythropoietin (EPO), osteoinductive factors, interferons such as interferon-alpha, beta, and -gamma, colony stimulating factors (CSFs) such as macrophage-CSF (M-CSF), granulocyte-macrophage-CSF (GM-CSF), and granulocyte-CSF (G-CSF), interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta, and other polypeptide factors including LIF and kit ligand (KL).

In various embodiments, the donor repair template comprises one or more polynucleotides encoding a gene or transgene selected from the group consisting of: a bispecific T cell engager (BiTE) molecule; a cytokine (e.g., IL-2, insulin, IFN-γ, IL-7, IL-21, IL-10, IL-12, IL-15, and TNF-α), a chemokine (e.g., MIP-1α, MIP-1β, MCP-1, MCP-3, and RANTES), a cytotoxin (e.g., Perforin, Granzyme A, and Granzyme B), a cytokine receptor (e.g., an IL-2 receptor, an IL-7 receptor, an IL-12 receptor, an IL-15 receptor, and an IL-21 receptor), and an engineered antigen receptor (e.g., an engineered T cell receptor (TCR), a chimeric antigen receptor (CAR), a Daric receptor or components thereof, or a chimeric cytokine receptor).

As used herein, the term “engineered TCR” refers to a T cell receptor, e.g., an αβ TCR that has a high-avidity and reactivity toward a target antigen. The engineered TCR may be selected, cloned, and subsequently introduced into a population of T cells used for adoptive immunotherapy. An engineered TCR is an exogenous TCR because it is introduced into T cells that do not normally express the particular TCR. The essential aspect of the engineered TCRs is that it has high avidity for a tumor antigen presented by a major histocompatibility complex (MHC) or similar immunological component. In contrast to engineered TCRs, CARS are engineered to bind target antigens in an MHC independent manner.

As used herein, the term “CAR” refers to a chimeric antigen receptor. Illustrative examples of CARs are disclosed in PCT Publication Nos.: WO2015164759, WO2015188119, and WO2016014789, each of which is incorporated herein by reference in its entirety.

As used herein, the term “Daric receptor” refers to a multichain engineered antigen receptor. Illustrative examples of Daric architectures and components thereof are disclosed in PCT Publication No. WO2015/017214 and U.S. Patent Publication No. 20150266973, each of which is incorporated herein by reference in its entirety.

As used herein, the terms “chimeric cytokine receptor” or “zetakine” refer to chimeric transmembrane immunoreceptors that comprise an extracellular domain comprising a soluble receptor ligand linked to a support region capable of tethering the extracellular domain to a cell surface, a transmembrane region and an intracellular signaling domain. Illustrative examples of zetakines are disclosed in U.S. Pat. Nos. 7,514,537; 8,324,353; 8,497,118; and 9,217,025, each of which is incorporated herein by reference in its entirety.

### G. Genome Edited Cells

The genome edited cells manufactured by the methods contemplated in particular embodiments provide improved gene therapy compositions. Without wishing to be bound to any particular theory, it is believed that the compositions and methods contemplated herein provide a more potent genome edited cell composition due to the increased genome editing efficiency achieved using the genome editing enhancers.

Genome edited cells contemplated in particular embodiments may be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are obtained from a mammalian subject. In a more preferred embodiment, the cells are obtained from a primate subject. In the most preferred embodiment, the cells are obtained from a human subject.

An “isolated cell” refers to a non-naturally occurring cell, e.g., a cell that does not exist in nature, a modified cell, an engineered cell, etc., that has been obtained from an in vivo tissue or organ and is substantially free of extracellular matrix.

Illustrative examples of cell types whose genome can be edited using the compositions and methods contemplated herein include, but are not limited to, cell lines, primary cells, stem cells, progenitor cells, and differentiated cells.

The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Symmetric cell division produces two identical daughter cells. “Proliferation” or “expansion” of cells refers to symmetrically dividing cells.

As used herein, the term “progenitor” or “progenitor cells” refers to cells have the capacity to self-renew and to differentiate into more mature cells. Many progenitor cells differentiate along a single lineage, but may have quite extensive proliferative capacity.

In one embodiment, the genome edited cell is an embryonic stem cell.

In one embodiment, the genome edited cell is an adult stem or progenitor cell.

In particular embodiments, the genome edited cell is a stem or progenitor cell selected from the group consisting of: mesodermal stem or progenitor cells, endodermal stem or progenitor cells, and ectodermal stem or progenitor cells.

In related embodiments, the genome edited cell is a mesodermal stem or progenitor cell. Illustrative examples of mesodermal stem or progenitor cells include, but are not limited to bone marrow stem or progenitor cells, umbilical cord stem or progenitor cells, adipose tissue derived stem or progenitor cells, hematopoietic stem or progenitor cells (HSPCs), mesenchymal stem or progenitor cells, muscle stem or progenitor cells, kidney stem or progenitor cells, osteoblast stem or progenitor cells, chondrocyte stem or progenitor cells, and the like.

In other related embodiments, the genome edited cell is an ectodermal stem or progenitor cell. Illustrative examples of ectodermal stem or progenitor cells include, but are not limited to neural stem or progenitor cells, retinal stem or progentior cells, skin stem or progenitor cells, and the like.

In other related embodiments, the genome edited cell is an endodermal stem or progenitor cell. Illustrative examples of endodermal stem or progenitor cells include, but are not limited to liver stem or progenitor cells, pancreatic stem or progenitor cells, epithelial stem or progenitor cells, and the like.

In one embodiment, the genome edited cell is a bone cell, osteocyte, osteoblast, adipose cell, chondrocyte, chondroblast, muscle cell, skeletal muscle cell, myoblast, myocyte, smooth muscle cell, bladder cell, bone marrow cell, central nervous system (CNS) cell, peripheral nervous system (PNS) cell, glial cell, astrocyte cell, neuron, pigment cell, epithelial cell, skin cell, endothelial cell, vascular endothelial cell, breast cell, colon cell, esophagus cell, gastrointestinal cell, stomach cell, colon cell, head cell, neck cell, gum cell, tongue cell, kidney cell, liver cell, lung cell, nasopharynx cell, ovary cell, follicular cell, cervical cell, vaginal cell, uterine cell, pancreatic cell, pancreatic parenchymal cell, pancreatic duct cell, pancreatic islet cell, prostate cell, penile cell, gonadal cell, testis cell, hematopoietic cell, lymphoid cell, or myeloid cell.

In a preferred embodiment, the genome editing compositions and methods are used to edit hematopoietic cells, e.g., hematopoietic stem cells, hematopoietic progenitor cells, immune effector cells, T cells, NKT cells, NK cells and the like.

Illustrative sources to obtain hematopoietic cells include, but are not limited to: cord blood, bone marrow or mobilized peripheral blood.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells can repopulate the erythroid, neutrophil-macrophage, megakaryocyte and lymphoid hematopoietic cell pool.

Additional illustrative examples of hematopoietic stem or progenitor cells suitable for use with the methods and compositions contemplated herein include hematopoietic cells that are CD34⁺CD38^{Lo}CD90⁺CD45^{RA-}, hematopoietic cells that are CD34⁺, CD59⁺, Thy1/CD90⁺, CD38^{Lo/-}, C-kit/CD117⁺, and Lin⁽⁻⁾, and hematopoietic cells that are CD133⁺.

In one embodiment, hematopoietic cells are CD34⁺CD133⁺ cells.

Various methods exist to characterize hematopoietic hierarchy. One method of characterization is the SLAM code. The SLAM (Signaling lymphocyte activation molecule) family is a group of >10 molecules whose genes are located mostly tandemly in a single locus on chromosome 1 (mouse), all belonging to a subset of immunoglobulin gene superfamily, and originally thought to be involved in T-cell stimulation. This family includes CD48, CD150, CD244, etc., CD150 being the founding member, and, thus, also called slamF1, i.e., SLAM family member 1. The signature SLAM code for the hematopoietic hierarchy is hematopoietic stem cells (HSC)—CD150⁺CD48⁻CD244⁻; multipotent progenitor cells (MPPs)—CD150⁻CD48⁻CD244⁺; lineage-restricted progenitor cells (LRPs)—CD150⁻CD48⁺CD244⁺; common myeloid progenitor (CMP)—lin-SCA-1-c-kit⁺CD34⁺CD16/32^{mid}; granulocyte-macrophage progenitor (GMP)—lin⁻SCA-1-c-kit⁺CD34⁺CD16/32^{hi}; and megakaryocyte-erythroid progenitor (MEP)—lin⁻SCA-1-c-kit⁺CD34⁻CD16/32^{low}.

In one embodiment, the hematopoietic cells are CD150⁺CD48⁻CD244⁻ cells.

In one embodiment, the hematopoietic cells are CD34⁺ hematopoietic cells.

In various embodiments, the hematopoietic cell is an immune effector cell. An “immune effector cell,” is any cell of the immune system that has one or more effector functions (e.g., cytotoxic cell killing activity, secretion of cytokines, induction of ADCC and/or CDC). Illustrative immune effector cells contemplated in particular embodiments are T lymphocytes, in particular cytotoxic T cells (CTLs; CD8⁺ T cells), TILs, and helper T cells (HTLs; CD4⁺ T cells). In one embodiment, immune effector cells include natural killer (NK) cells. In one embodiment, immune effector cells include natural killer T (NKT) cells.

The terms “T cell” or “T lymphocyte” are art-recognized and are intended to include thymocytes, naïve T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, CD4⁻CD8⁻ T cell, or any other subset of T cells. In one embodiment, the T cell is an NKT cell. Other illustrative populations of T cells suitable for use in particular embodiments include naïve T cells and memory T cells.

“Potent T cells,” and “young T cells,” are used interchangeably in particular embodiments and refer to T cell phenotypes wherein the T cell is capable of proliferation and a concomitant decrease in differentiation. In particular embodiments, the young T cell has the phenotype of a “naïve T cell.” In particular embodiments, young T cells comprise one or more of, or all of the following biological markers: CD62L, CCR7, CD28, CD27, CD122, CD127, CD197, and CD38. In one embodiment, young T cells comprise one or more of, or all of the following biological markers: CD62L, CD127, CD197, and CD38. In one embodiment, the young T cells lack expression of CD57, CD244, CD160, PD-1, CTLA4, TIM3, and LAG3.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors.

### H. Polypeptides

Various polypeptides are contemplated herein, including, but not limited to, meganucleases, megaTALs, TALENs, ZFNs, Cas nucleases, end-processing nucleases, engineered antigen receptors, therapeutic polypeptides, fusion polypeptides, and vectors that express polypeptides. “Polypeptide,” “polypeptide fragment,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well-known recombinant and/or synthetic techniques. Polypeptides are not limited to a specific length, e.g., they may comprise a full length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances.

Illustrative examples of polypeptides contemplated in particular embodiments include, but are not limited to meganucleases, megaTALs, TALENs, ZFNs, Cas nucleases, end-processing nucleases, engineered TCRs, CARS, Darics, therapeutic polypeptides and fusion polypeptides and variants thereof.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to improve the biological properties of engineered nuclease, engineered TCR, CAR, Daric or the like by introducing one or more substitutions, deletions, additions and/or insertions into the polypeptide. In particular embodiments, polypeptides include polypeptides having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to any of the reference sequences contemplated herein, typically where the variant maintains at least one biological activity of the reference sequence.

Polypeptides variants include biologically active “polypeptide fragments.” As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. Polypeptide fragments refer to a polypeptide, which can be monomeric or multimeric that has an amino-terminal deletion, a carboxyl-terminal deletion, and/or an internal deletion or substitution of one or more amino acids of a naturally-occurring or recombinantly-produced polypeptide. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long.

Polypeptides contemplated in particular embodiments include fusion polypeptides.

Illustrative examples of fusion proteins contemplated in particular embodiments, polypeptides include polypeptides having at least about include, but are not limited to: megaTALs, TALENs, ZFNs, Cas nucleases, end-processing nucleases, engineered antigen receptors, and other polypeptides.

Fusion polypeptides may optionally comprise a linker that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions.

Exemplary linkers include, but are not limited to the following amino acid sequences: glycine polymers (G)ₙ; glycine-serine polymers (G₁₋₅S₁₋₅)ₙ, where n is an integer of at least one, two, three, four, or five; glycine-alanine polymers; alanine-serine polymers; GGG (SEQ ID NO: 1); DGGGS (SEQ ID NO: 2); TGEKP (SEQ ID NO: 3) (see e.g., Liu et al., PNAS 5525-5530 (1997)); GGRR (SEQ ID NO: 4) (Pomerantz et al. 1995, supra); (GGGGS)ₙ wherein n=1, 2, 3, 4 or 5 (SEQ ID NO: 5) (Kim et al., PNAS 93, 1156-1160 (1996.); EGKSSGSGSESKVD (SEQ ID NO: 6) (Chaudhary et al., 1990, Proc. Natl. Acad. Sci. U.S.A. 87:1066-1070); KESGSVSSEQLAQFRSLD (SEQ ID NO: 7) (Bird et al., 1988, Science 242:423-426), GGRRGGGS (SEQ ID NO: 8); LRQRDGERP (SEQ ID NO: 9); LRQKDGGGSERP (SEQ ID NO: 10); LRQKD(GGGS)₂ERP (SEQ ID NO: 11). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein or between an endogenous open reading frame and a polypeptide encoded by a donor repair template. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 12), for example, ENLYFQG (SEQ ID NO: 13) and ENLYFQS (SEQ ID NO: 14), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

### I. Polynucleotides

In particular embodiments, polynucleotides encoding one or more meganucleases, megaTALs, TALENs, ZFNs, Cas nucleases, end-processing nucleases, engineered TCRs, CARS, Darics, therapeutic polypeptides, fusion polypeptides contemplated herein are provided.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (gRNA), plus strand RNA (RNA(+)), minus strand RNA (RNA(−)), tracrRNA, crRNA, single guide RNA (sgRNA), synthetic RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

In particular embodiments, polynucleotides may be codon-optimized. As used herein, the term “codon-optimized” refers to substituting codons in a polynucleotide encoding a polypeptide in order to increase the expression, stability and/or activity of the polypeptide.

As used herein, the terms “polynucleotide variant” and “variant” and the like refer to polynucleotides displaying substantial sequence identity with a reference polynucleotide sequence or polynucleotides that hybridize with a reference sequence under stringent conditions. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant polynucleotide, a synthetic polynucleotide, or other polynucleotide that does not exist in nature and that has been made by the hand of man.

The term “nucleic acid cassette” or “expression cassette” as used herein refers to genetic sequences within a vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. In particular embodiments, a donor repair template comprises one or more nucleic acid cassettes.

As used herein, the term “polynucleotide(s)-of-interest” refers to one or more polynucleotides, e.g., a polynucleotide encoding a polypeptide (i.e., a polypeptide-of-interest), inserted into an expression vector.

In a certain embodiment, a polynucleotide-of-interest comprises an inhibitory polynucleotide including, but not limited to, a crRNA, a tracrRNA, a single guide RNA (sgRNA), an siRNA, an miRNA, an shRNA, a ribozyme or another inhibitory RNA.

Polynucleotides, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, post-transcription response elements, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector.

In particular embodiments, the vector integrates into a cell's genome.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

Illustrative examples of vectors include, but are not limited to plasmid, autonomously replicating sequences, and transposable elements, e.g., Sleeping Beauty, PiggyBac.

Additional illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a cell.

Illustrative methods of delivering polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

Polynucleotides encoding one or more therapeutic polypeptides, or fusion polypeptides may be introduced into a target cell by viral methods.

### J. Viral Vectors

In particular embodiments, polynucleotides are introduced into a target cell using a vector, preferably a viral vector, more preferably a retroviral vector, and even more preferably, a lentiviral vector.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a virus or viral particle that mediates nucleic acid transfer. Viral particles will typically include various viral components and sometimes also host cell components in addition to nucleic acid(s).

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors comprising engineered nucleases and/or donor repair templates are administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include, but are not limited to adeno-associated virus (AAV), retrovirus, herpes simplex virus, adenovirus, vaccinia virus vectors for gene transfer.

In various embodiments, one or more polynucleotides encoding an engineered nuclease and/or donor repair template are introduced into a cell by transducing the cell with a recombinant adeno-associated virus (rAAV), comprising the one or more polynucleotides. AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10. In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In some embodiments, engineering and selection methods can be applied to AAV capsids to make them more likely to transduce cells of interest. Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides encoding an engineered nuclease and/or donor repair template are introduced into a cell by transducing the cell with a retrovirus, e.g., lentivirus, comprising the one or more polynucleotides.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (Ψ) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding an engineered nuclease and/or donor repair template are introduced into a cell by transducing the cell with an adenovirus comprising the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding an engineered nuclease and/or donor repair template are introduced into a cell by transducing the cell with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### K. Compositions and Formulations

The compositions contemplated in particular embodiments may comprise one or more polypeptides, polynucleotides, vectors comprising same, and genome editing compositions and genome edited cell compositions, as contemplated herein.

The genome editing compositions and methods contemplated in particular embodiments are useful for editing a population of cells. As used herein, the term “population of cells” refers to a plurality of cells that may be made up of any number and/or combination of homogenous or heterogeneous cell types, as described elsewhere herein. For example, a population of cells may comprise about 10%, about 20%, about 30%, about 40%, about 50%, about 60%, about 70%, about 80%, about 90%, or about 100% of the target cell type to be transduced.

In various embodiments, the compositions contemplated herein comprise a genome editing enhancer, and an engineered nuclease. The engineered nuclease may be in the form of an mRNA that is introduced into the cell via polynucleotide delivery methods disclosed supra, e.g., electroporation, lipid nanoparticles, etc. The composition may be used to generate an enhanced population of genome edited cells with an increased or enhanced rate of genome editing by error prone NHEJ, compared to a composition lacking a genome editing enhancer.

In various embodiments, the compositions contemplated herein comprise a genome editing enhancer, a donor repair template and an engineered nuclease. The engineered nuclease may be in the form of an mRNA that is introduced into the cell via polynucleotide delivery methods disclosed supra, e.g., electroporation, lipid nanoparticles, etc. The composition may be used to generate an enhanced population of genome edited cells with an increased or enhanced rate of genome editing by HDR, compared to a composition lacking a genome editing enhancer.

In particular embodiments, the compositions contemplated herein comprise a population of cells, a genome editing enhancer, an engineered nuclease, and optionally, a donor repair template. The engineered nuclease may be in the form of an mRNA that is introduced into the cell via polynucleotide delivery methods disclosed supra.

In particular embodiments, the population of cells comprise hematopoietic cells including, but not limited to, hematopoietic stem cells, hematopoietic progenitor cells, and T cells.

In particular embodiments, the genome editing enhancer is preferably a nucleic acid intercalator, more preferably a DNA intercalator, even more preferably an acridine, and even more preferably 9-aminoacridine.

Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount genome edited cells manufactured by the methods contemplated herein comprising a genome editing enhancer. In preferred embodiments, the pharmaceutical cell compositions comprise a population of cells comprising an increased proportion of genome edited cells compared to a population of cells that has not been edited using a genome editing enhancer.

In certain embodiments, the pharmaceutical cell compositions manufactured using a genome editing enhancer comprises a population of cells comprising about 50%, about 55%, about 60%, about 65%, about 70%, about 75%, about 80%, about 85%, about 90%, about 95%, or about 96%, 97%, 98%, or 99% genome edited cells.

It can generally be stated that a pharmaceutical composition comprising the genome edited cells manufactured by the methods contemplated in particular embodiments may be administered at a dosage of about 10² to about 10¹⁰ cells/kg body weight, about 10⁵ to about 10⁹ cells/kg body weight, about 10⁵ to about 10⁸ cells/kg body weight, about 10⁵ to about 10⁷ cells/kg body weight, about 10⁷ to about 10⁹ cells/kg body weight, or about 10⁷ to about 10⁸ cells/kg body weight, including all integer values within those ranges. The number of cells will depend upon the percentage of genome edited cells in the compositions, the ultimate use for which the composition is intended, as well as the type of cells included therein. For uses provided herein, the cells are generally in a volume of a liter or less, can be 500 mL or less, even 250 mL or 100 mL or less. Hence the density of the desired cells is typically greater than about 10⁶ cells/mL and generally is greater than about 10⁷ cells/mL, generally about 10⁸ cells/mL or greater. The clinically relevant number of cells can be apportioned into multiple infusions that cumulatively equal or exceed about 10⁵, 10⁶, 10⁷, 10⁸, 10⁹, 10¹⁰, 10¹¹, or 10¹² cells.

In some embodiments, particularly since there is a high proportion of genome edited cells in the population of cells, lower numbers of cells, in the range of 10⁶/kilogram (10⁶-10¹¹ per patient) may be administered multiple times at dosages within these ranges. The cells may be allogeneic, syngeneic, xenogeneic, or autologous to the patient undergoing therapy.

In particular embodiments, pharmaceutical compositions contemplated herein comprise an amount of genome edited T cells, in combination with one or more pharmaceutically or physiologically acceptable carriers, diluents or excipients.

Pharmaceutical compositions comprising genome edited cells contemplated in particular embodiments may further comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions contemplated in particular embodiments are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the genome edited cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising genome edited cells contemplated herein are formulated in a solution comprising PlasmaLyte A.

In another preferred embodiment, compositions comprising genome edited cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising genome edited cells contemplated herein are formulated in a solution comprising 50:50 PlasmaLyte A to CryoStor CS10.

In a particular embodiment, compositions contemplated herein comprise an effective amount of a genome edited cell composition, alone or in combination with one or more therapeutic agents. Thus, the compositions may be administered alone or in combination with other known treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated in particular embodiments include cytokines, growth factors, steroids, NSAIDs, DMARDs, anti-inflammatories, chemotherapeutics, radiotherapeutics, therapeutic antibodies, or other active and ancillary agents.

### L. Genome Editing Methods

In various embodiments, methods of editing the genome of a population of cells is contemplated. The genome editing compositions and methods of using the same to edit the genome of cells provide increased genome editing efficiency. Without wishing to be bound to any particular theory, it is contemplated that use of the genome editing enhancers in the genome editing compositions contemplated herein, significantly increases the number of genome edited cells in a population.

In particular embodiments, the genome editing enhancers increase the proportion of genome edited cells in a population about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, or more, compared to the number of genome edited cells in a population manufactured without the use of a genome editing enhancer. In particular embodiments, the genome editing enhancers increase the proportion of genome edited cells in a population about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold or more, compared to the number of genome edited cells in a population manufactured without the use of a genome editing enhancer.

Genome editing methods contemplated in particular embodiments comprise introducing one or more engineered nucleases and a genome editing enhancer contemplated herein into a population of cells in order to create a DSB at a target site and optionally introducing an end-processing enzyme such as a 3′ to 5′ exonuclease or biologically active fragment thereof, e.g., Trex2, into the cell to increase the rate or frequency of repair of the break by error-prone NHEJ.

Genome editing methods contemplated in particular embodiments comprise introducing one or more engineered nucleases and a genome editing enhancer contemplated herein into a population of cells in order to create a DSB at a target site and subsequently introducing one or more donor repair templates into the population of cells that will be incorporated into the cell's genome at the DSB site by homologous recombination.

In one embodiment, methods of increasing genome editing in a population of cells comprises introducing an engineered nuclease into a cell, contacting the cell with a genome editing enhancer to increase the frequency of genome editing in the population of cells.

In one embodiment, methods of increasing homology directed repair (HDR) in a population of cells comprises introducing an engineered nuclease and a donor repair template into a cell, and contacting the cell with a genome editing enhancer to increase the frequency of HDR in the population of cells.

In one embodiment, methods of increasing non-homologous end joining (NHEJ) in a population of cells comprises introducing an engineered nuclease and optionally an end-processing enzyme, e.g., a 3′ to 5′ exonuclease (Trex2, Exo1, TdT etc.) into a cell, and contacting the cell with a genome editing enhancer to increase the frequency of NHEJ in the population of cells.

Genome editing enhancers can be used at any suitable concentration in particular embodiments, so long as the rate or efficiency of genome editing is increased. In particular embodiments, a genome editing enhancer is used to increase genome editing at a concentration of about 0.01 μM to about 200 μM, about 0.01 μM to about 100 μM, about 0.01 μM to about 10 μM, about 0.01 μM to about 1.0 μM, about 0.1 μM to about 200 μM, about 0.1 μM to about 100 μM, about 0.1 μM to about 10 μM, about 0.1 μM to about 1.0 μM, about 1.0 μM to about 200 μM, about 1.0 μM to about 100 μM, about 1.0 μM to about 10 μM, or about 0.01 μM, about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 2.0 μM, about 3.0 μM, about 4.0 μM, about 5.0 μM, about 6.0 μM, about 7.0 μM, about 8.0 μM, about 9.0 μM, about 10 μM, about 20 μM, about 30 μM, about 40 μM, about 50 μM, about 60 μM, about 70 μM, about 80 μM, about 90 μM, or about 100 μM or higher and any intervening concentration thereof.

In particular embodiments, the one or more nucleases are introduced into a cell using a vector. In other embodiments, the one or more nucleases are preferably introduced into a cell as mRNAs. The nucleases may be introduced into the cells by microinjection, transfection, lipofection, heat-shock, electroporation, transduction, gene gun, microinjection, DEAE-dextran-mediated transfer, and the like.

In a particular embodiment, one or more donor templates comprising a polynucleotide encoding a therapeutic gene or fragment thereof, transgene, or selectable marker.

In various embodiments, the donor repair template comprises one or more polynucleotides encoding a gene or fragment thereof including, but not limited to: β globin, δ globin, γ globin, BCL11A, KLF1, CCR5, CXCR4, PPP1R12C (AAVS1), HPRT, albumin, Factor VIII, Factor IX, LRRK2, Htt, SOD1, C9orf72, TARDBP, FUS, RHO, CFTR, SFTPB, TRAC, TRBC, PD1, CTLA-4, HLA A, HLA B, HLA C, HLA-DP, HLA-DQ, HLA-DR, LMP7, TAP 1, TAP2, TAPBP, CIITA, DMD, GR, IL2RG, Rag-1, RFX5, FAD2, FAD3, ZP15, KASII, MDH, and EPSPS; a bispecific T cell engager (BiTE) molecule; a hormone; a cytokine (e.g., IL-2, insulin, IFN-γ, IL-7, IL-21, IL-10, IL-12, IL-15, and TNF-α), a chemokine (e.g., MIP-1α, MIP-1β, MCP-1, MCP-3, and RANTES), a cytotoxin (e.g., Perforin, Granzyme A, and Granzyme B), a cytokine receptor (e.g., an IL-2 receptor, an IL-7 receptor, an IL-12 receptor, an IL-15 receptor, and an IL-21 receptor), and an engineered antigen receptor (e.g., an engineered T cell receptor (TCR), a chimeric antigen receptor (CAR), a Daric receptor or components thereof, or a chimeric cytokine receptor).

The donor templates may be introduced into the cells by microinjection, transfection, lipofection, heat-shock, electroporation, transduction, gene gun, microinjection, DEAE-dextran-mediated transfer, and the like.

In a preferred embodiment, the one or more nucleases are introduced into the cell by mRNA electroporation and the one or more donor repair templates are introduced into the cell by viral transduction.

In another preferred embodiment, the one or more nucleases are introduced into the cell by mRNA electroporation and the one or more donor repair templates are introduced into the cell by AAV transduction. The AAV vector may comprise ITRs from AAV2, and a serotype from any one of AAV1, AAV2, AAV3, AAV4, AAV5, AAV6, AAV7, AAV8, AAV9, or AAV10. In preferred embodiments, the AAV vector may comprise ITRs from AAV2 and a serotype from AAV2 or AAV6.

In another preferred embodiment, the one or more nucleases are introduced into the cell by mRNA electroporation and the one or more donor repair templates are introduced into the cell by lentiviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV).

The population of cells may be contacted with a genome editing enhancer before or during, or after introduction of one or more engineered nucleases and a donor repair template are introduced into the cells. The one or more donor repair templates may be delivered prior to, simultaneously with, or after the one or more engineered nucleases and genome editing enhancers are introduced into a cell. In certain embodiments, the one or more donor repair templates are delivered simultaneously with the one or more engineered nucleases and the genome editing enhancer. In other embodiments, the one or more donor repair templates are delivered prior to the one or more engineered nucleases and the genome editing enhancer, for example, seconds to hours to days before the one or more donor repair templates, including, but not limited to about 1 min. to about 30 min., about 1 min. to about 60 min., about 1 min. to about 90 min., about 1 hour to about 24 hours before the one or more engineered nucleases or more than 24 hours before the one or more engineered nucleases. In certain embodiments, the one or more donor repair templates are delivered after the nuclease and the genome editing enhancer, preferably within about 1, 2, 3, 4, 5, 6, 7, or 8 hours; more preferably, within about 1, 2, 3, or 4 hours; or more preferably, within about 4 hours.

The one or more donor repair templates may be delivered using the same delivery systems as the one or more engineered nucleases. By way of non-limiting example, when delivered simultaneously, the donor repair templates and engineered nucleases may be encoded by the same vector, e.g., an IDLV lentiviral vector or an AAV vector (e.g., AAV6). In particular preferred embodiments, the engineered nuclease(s) are delivered by mRNA electroporation and the donor repair templates are delivered by transduction with an AAV vector.

In particular embodiments, where a CRISPR/Cas nuclease system is used to modify a target site in a cell, the Cas nuclease is introduced into the cell by mRNA electroporation and an expression cassette encoding a tracrRNA:crRNA or sgRNA that binds near the site to be edited in the genome and donor repair template are delivered by transduction with an IDLV lentiviral vector or an AAV vector.

In particular embodiments, where a CRISPR/Cas nuclease system is used to modify a target site in a cell, the Cas nuclease and the tracrRNA:crRNA or sgRNA that binds near the site to be edited in the genome are introduced into the cell by mRNA electroporation and the donor repair template is delivered by transduction with an IDLV lentiviral vector or an AAV vector.

In one embodiment, the tracrRNA:crRNA or the sgRNA are chemically synthesized RNA, that have chemically protected 5 and 3′ ends.

In another embodiment, Cas9 is delivered as protein complexed with chemically synthesized tracrRNA:crRNA or sgRNA.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### A Small Molecule Screen Identifies Gene Editing Enhancers

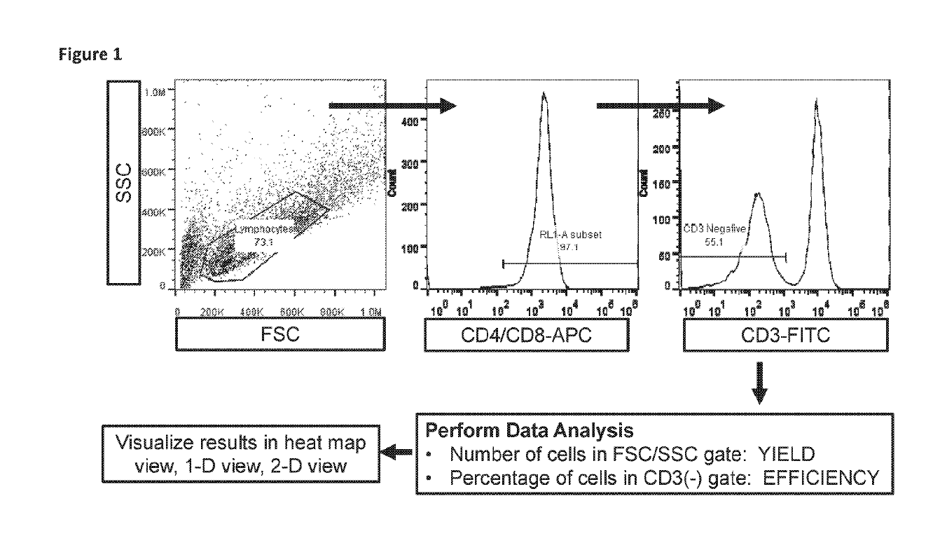

A small molecule screen (FIG. 1) was performed to identify candidate soluble factors which could modulate the efficiency of genomic editing at the TCR locus in primary human T cells.

Briefly, purified CD3+ primary human T cells were activated with CD3/CD28 magnetic beads and cultured for 48 hours in IL-2 supplemented complete RPMI media prior to bead removal. After bead removal, T cells were washed and electroporated with in vitro transcribed mRNA encoding a T cell receptor alpha (TCRα) targeting megaTAL-Trex2 fusion mRNA. Cells were then distributed into 384-well format and contacted with a library of ˜2000 known drugs, natural products, and other bioactive components (Microsource Discovery System's Spectrum Collection), with each drug at a final concentration of 10 μM. Cells were cultured with the compounds for 24 hours at 37° C. and then washed and cultured for an additional three days. Cells were then stained for CD3, CD4, and CD8 surface markers, and subjected to high-throughput volumetric flow cytometric analysis. MegaTAL mediated disruption of the TCRα gene was detected by loss of CD3 cell surface staining.

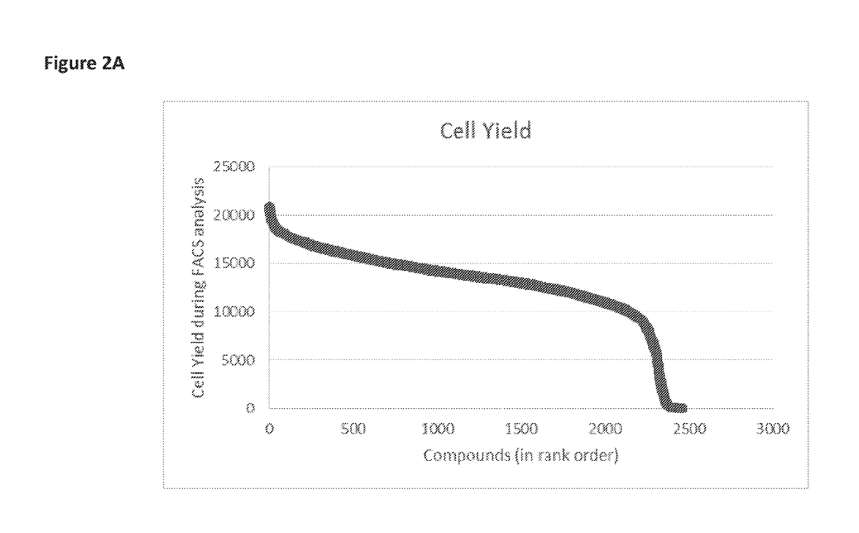

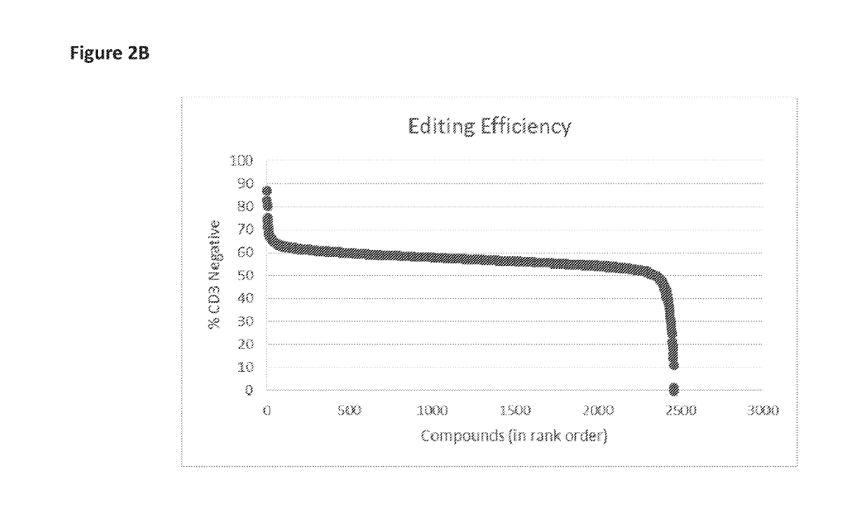

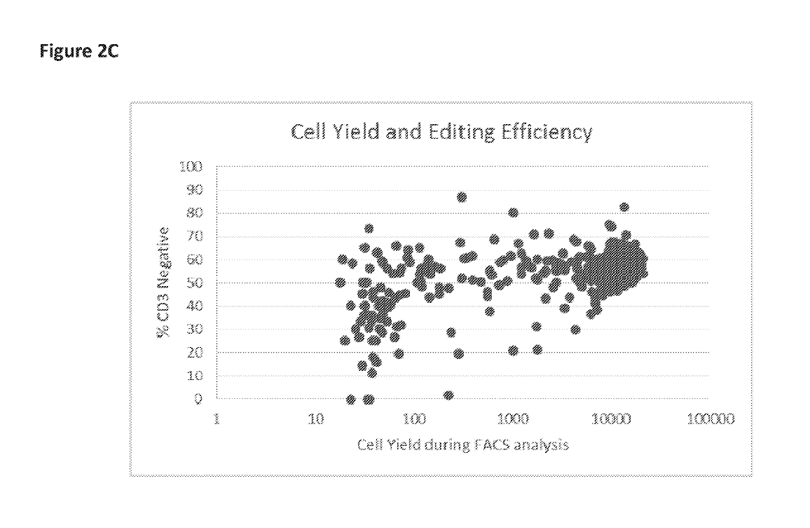

MegaTAL editing efficiency in the presence of each compound was quantified as the proportion of CD4/CD8+ cells which stained negative for CD3. The number of flow cytometry events in the “live” gate of the FSC/SSC profile was used as a measure of cell yield. The effects of each compound on genome editing was analyzed by plotting the compounds in rank order according to effect on cell yield (FIG. 2A), according to the frequency of CD3-negative cells (FIG. 2B) at the time of flow cytometry analysis, and by plotting the frequency of CD3-negative cells as a function of cell yield for all 2000 compounds (FIG. 2C). The yield and megaTAL activity for the ten most active compounds is shown in Table 1.

TABLE 1
Compounds that enhance genome editing.

| | | | % CD3 |
|---|---|---|---|
| Compound | [Compound] | Cell Yield | negative |
| Tilorone | 10 μM | 310 | 86.8 |
| Aminacrine | 10 μM | 13621 | 82.8 |
| Homidium Bromide | 10 μM | 1023 | 80.3 |
| Harmine | 10 μM | 9581 | 75.1 |
| Hycanthone | 10 μM | 10012 | 74.1 |
| Daunorubicin | 10 μM | 35 | 73.3 |
| Sanguinarine Sulfate | 10 μM | 2346 | 71.1 |
| Kinetin Riboside | 10 μM | 1668 | 70.8 |
| Ethacridine Lactate | 10 μM | 14179 | 70.4 |
| Cyclohexamide | 10 μM | 4166 | 68.7 |

### Example 2

### Tilorone, Aminacrine, Homidium Bromide (Ethidium Bromide) and Harmine Enhance MegaTAL-Mediated Gene Disruption in Primary T Cells

T cells were purified, activated and electroporated with mRNA encoding TCRα-targeting megaTAL-Trex2 fusion as described in Example 1. Electroporated cells were cultured for 24 hours at either 30° C. or 37° C. with Tilorone, Aminacrine, Homidium Bromide (Ethidium Bromide) and Harmine. Cells were then washed to remove residual compound and cultured for an additional three days. After the three days of culture, the cells were stained with CD4, CD8 and CD3 and analyzed by flow cytometery as described in Example 1.

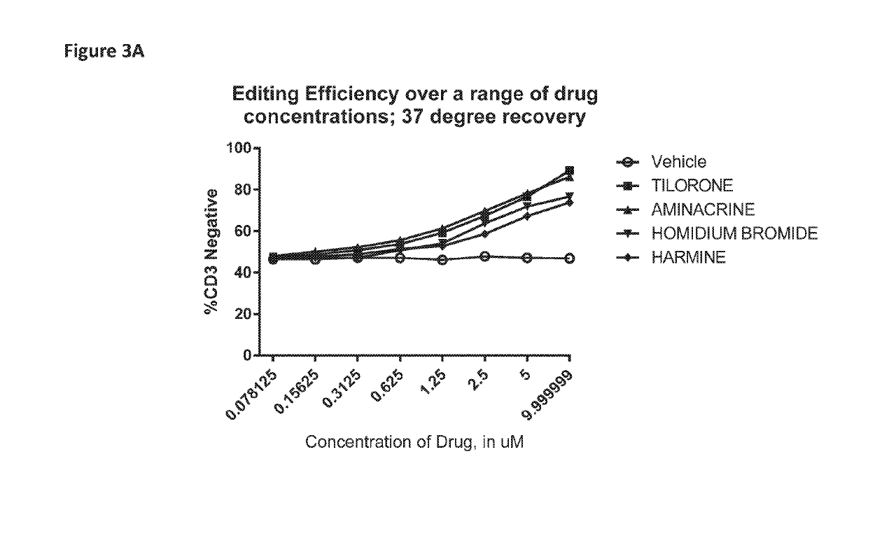

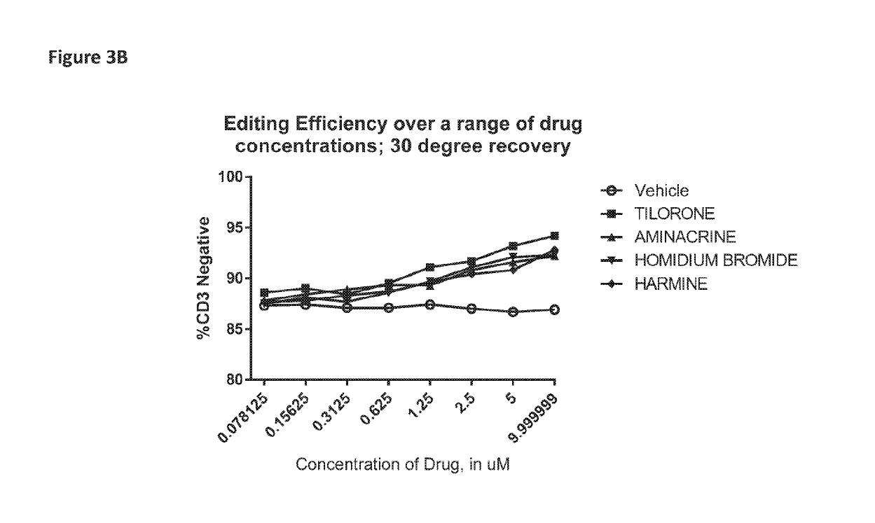

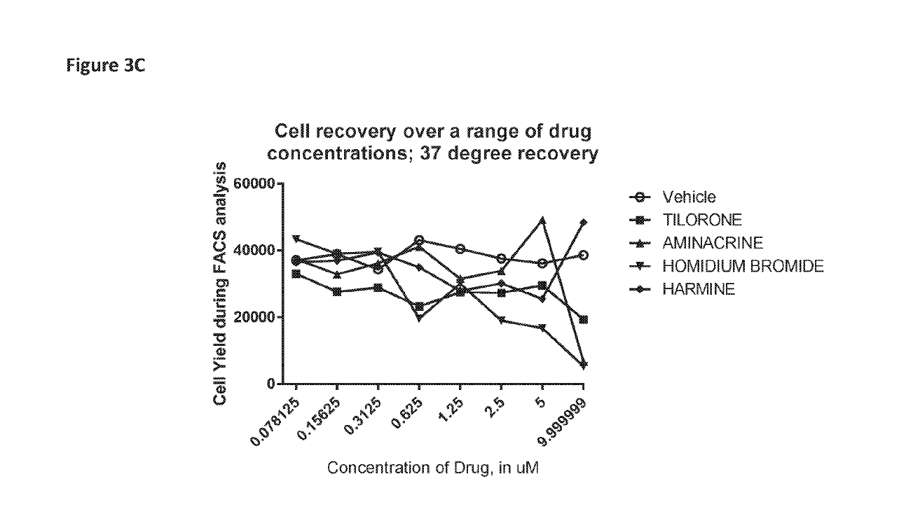

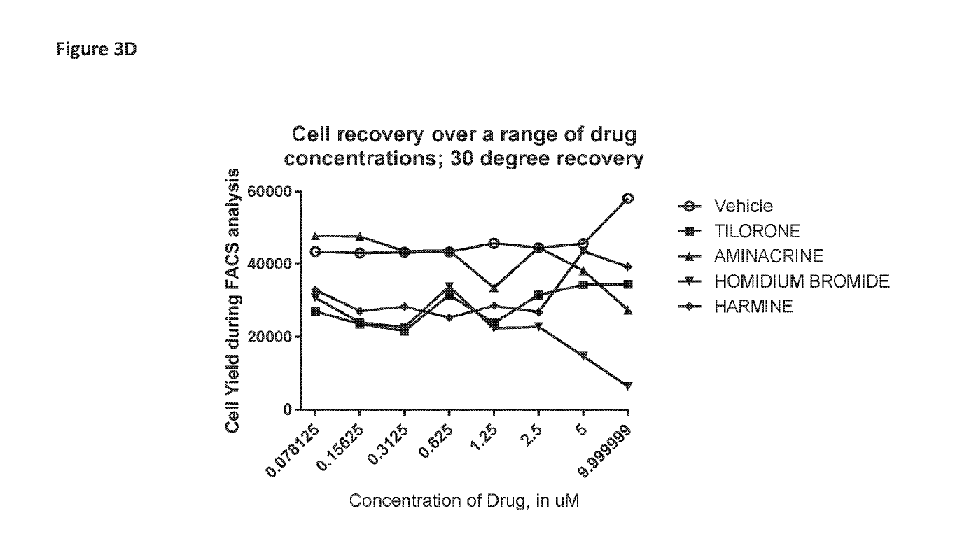

A positive correlation was observed between the concentration of each compound and the efficiency of TCRα disruption, as determined by proportion of CD3-negative cells. FIGS. 3A and 3B. In addition to concentration-dependent increase in gene disruption, each compound demonstrated a concentration-dependent impact on T cell viability and yield. FIGS. 3C and 3D. Aminacrine had minimal impact on cell viability and substantially enhanced gene disruption.

### Example 3

### Aminacrine Enhances Homologous Recombination of a Transgene Encoding a Fluorescent Protein into the T Cell Receptor Alpha (TCR_{A}) Locus

Adeno-associated virus (AAV) plasmids containing transgene cassettes comprising a promoter, a transgene encoding a fluorescent protein, and a polyadenylation signal were designed and constructed. The integrity of AAV ITR elements was confirmed with XmaI digest. The transgene expression cassette was placed between two homology regions within exon 1 of the TCRα gene to enable targeting by homologous recombination (AAV targeting vector) using a TCRα-targeting megaTAL. The 5′ and 3′ homology regions were ˜1500 bp and ˜1000 bp in length, respectively, and neither homology region contained the complete megaTAL target site. The transgene expression cassette contained a myeloproliferative sarcoma virus enhancer, negative control region deleted, d1587rev primer-binding site substituted (MND) promoter operably linked to a polynucleotide encoding a fluorescent polypeptide, e.g., green fluorescent protein (GFP). The expression cassettes also contain the SV40 late polyadenylation signal.

Recombinant AAV-6 (rAAV) was prepared by transiently co-transfecting HEK 293T cells with one or more plasmids providing the replication, capsid, and adenoviral helper elements necessary. rAAV was purified from the co-transfected HEK 293T cell culture using ultracentrifugation in an iodixanol-based gradient.

MegaTAL-induced homology directed repair (HDR) was evaluated in primary human T cells activated with CD3 and CD28 and cultured in complete media supplemented with IL-2. After 3 days, T cells were washed and electroporated with in vitro transcribed mRNA encoding a TCRα targeting megaTAL, and subsequently transduced with purified recombinant AAV encoding MND-GFP transgene cassette. Controls included T cells containing megaTAL or rAAV targeting vector alone. Cells were cultured overnight at 30° C. in the presence or absence of two different doses of aminacrine. Five days post electroporation the frequency of GFP+ T cells was measured by flow cytometry. MegaTAL mediated disruption of the TCRα gene was detected by loss of CD3 staining.

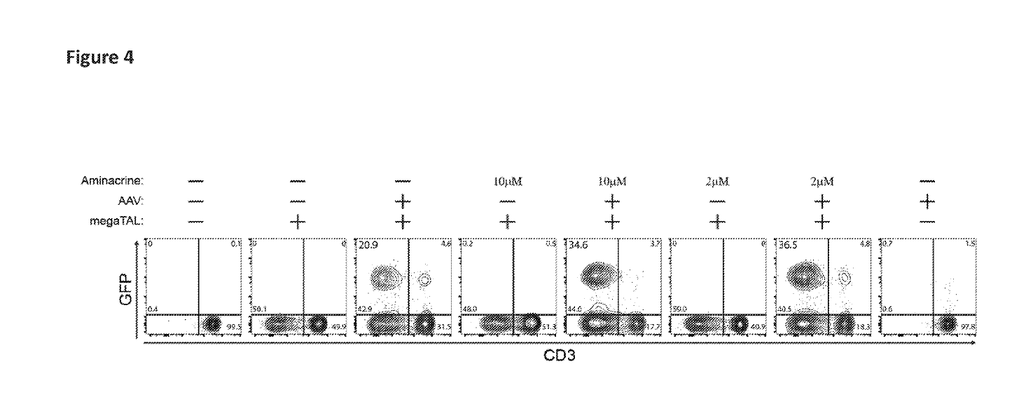

A concentration-dependent increase in the frequency of HDR events (% GFP+ cells) was observed when T cells were supplemented with aminacrine following megaTAL transfection (18% vs. 36% GFP+ with 10 μM aminacrine), whereas only minimal GFP expression was observed in samples treated with AAV alone. FIG. 4 and Table 2.

TABLE 2
Aminacrine elevates HDR levels in primary T cells.
Aminacrine HDR Assay

| megaTAL | − | + | + | + | + | + | + | − |
|---|---|---|---|---|---|---|---|---|
| Aminacrine | − | − | − | 10 μM | 10 μM | 2 μM | 2 μM | − |
| AAV-GFP | − | − | + | − | + | − | + | + |
| Donor 783 | 0 | 0 | 18% | 0 | 36% | 0 | 25% | 1.3% |
| Donor 855 | 0 | 0 | 26% | 0 | 38% | 0 | 41% | 1.2% |

Percentages show % GFP positive cells. (+) means that component was present, (−) means that component was absent.

### Example 4

### Aminacrine Enhances Homologous Recombination of a Transgene Encoding a Fluorescent Protein into the BCL11A Erythroid Enhancer Region

Adeno-associated virus (AAV) plasmids containing a promoter, a GFP or BFP reporter transgene and a polyadenylation signal were designed, constructed, and verified. The transgene was flanked by either 1.3 kb and 1.0 kb homology arms to the BCL11A erythroid enhancer locus at DHS58 (Bauer et al., Science. 342(6155): 253-7 (2013)). rAAV is generated by transient transfection of HEK293T cells, as described in Example 3.

Primary human peripheral blood-derived CD34+ cells were cultured in cytokine-supplemented media for 72 hours. Cells were washed and electroporated with either an mRNA encoding a megaTAL specific for the human BCL11A erythroid enhancer region or a megaTAL targeting the human CCR5 locus. Cells were then incubated with AAV vector with or without aminacrine at concentrations of 0 μM, 0.3 μM, 1.0 μM, and 3.0 μM for 24 hours, washed and cultured in cytokine-supplemented media. HDR was analyzed at regular timepoints by flow cytometry to determine BFP fluorescence.

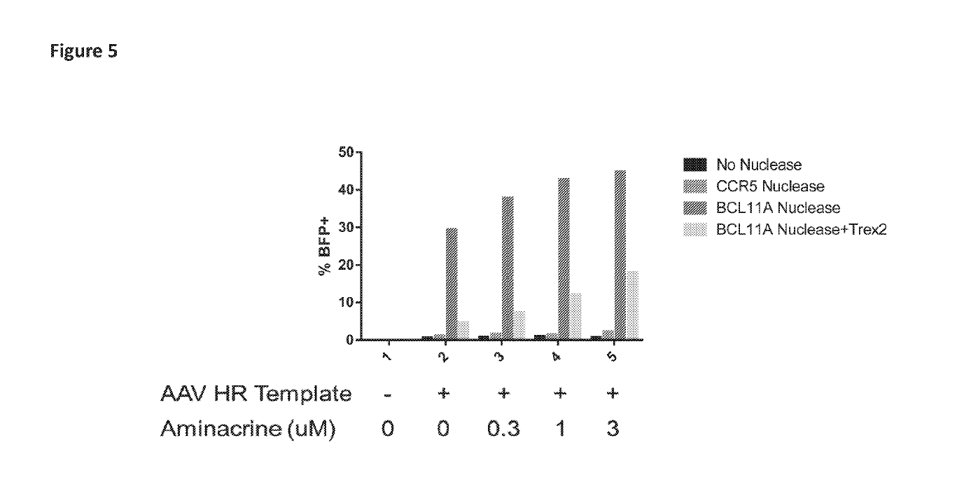

Aminacrine induced a concentration-dependent increase in HDR frequency in CD34+ cells at the BCL11A locus (% BFP+ cells). FIG. 5. Template capture via a non-homology driven NHEJ pathway in CD34+ cells was not enhanced by aminacrine. Aminacrine addition did not significantly increase the proportion of BFP+ cells when a CCR5-specific megaTAL was combined with the BCL11A-targeting AAV template. Id.

### Example 5

### Aminacrine Treatment does not Adversely Affect Hematopoietic Stem and Progenitor Cell Survival

A potential caveat with using small molecules to enhance gene repair is the impact on hematopoietic stem and progenitor cell survival. The methylceulluose colony forming assay was used to determine the impact of aminacrine treatment on hematopoietic stem and progenitor cell survival in vitro. Human peripheral blood CD34+ cells were cultured in cytokine supplemented media, electroporated with mRNA encoding BCL11A-specific megaTAL and transduced with BCL11A-HDR AAV vector, as described in Example 4.

Cells were cultured in the presence or absence of aminacrine during the first 24 hours post-electroporation. Following this post-electroporation recovery step, cells were counted and plated into methylcellulose media.

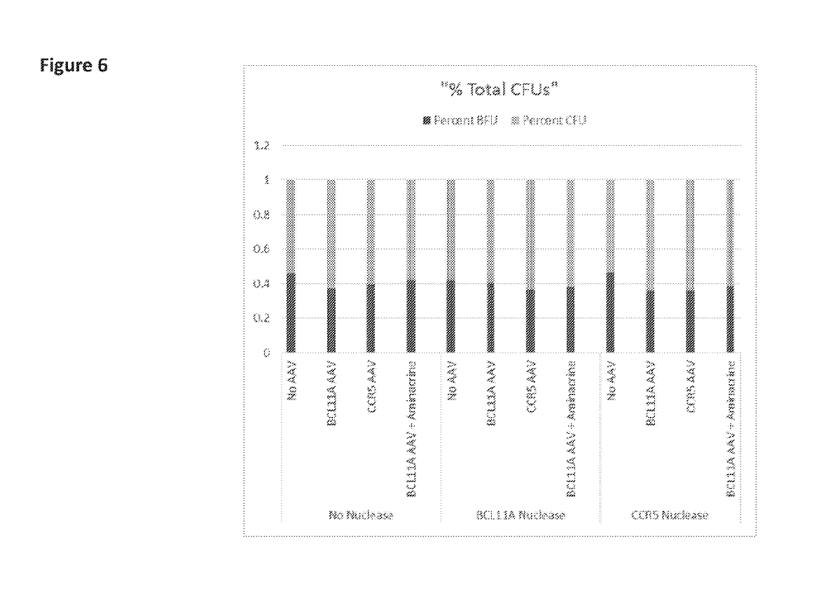

After 14 days in methylcellulose culture, the erythroid burst-forming units (BFU) and hematopoietic colony forming units (CFU) were scored based on frequency and morphology. The CD34+ cells treated with megaTAL alone, megaTAL with rAAV, with or without aminacrine, yielded comparable lineage output and showed no overt lineage skewing relative to control-electroporated cells. FIG. 6.

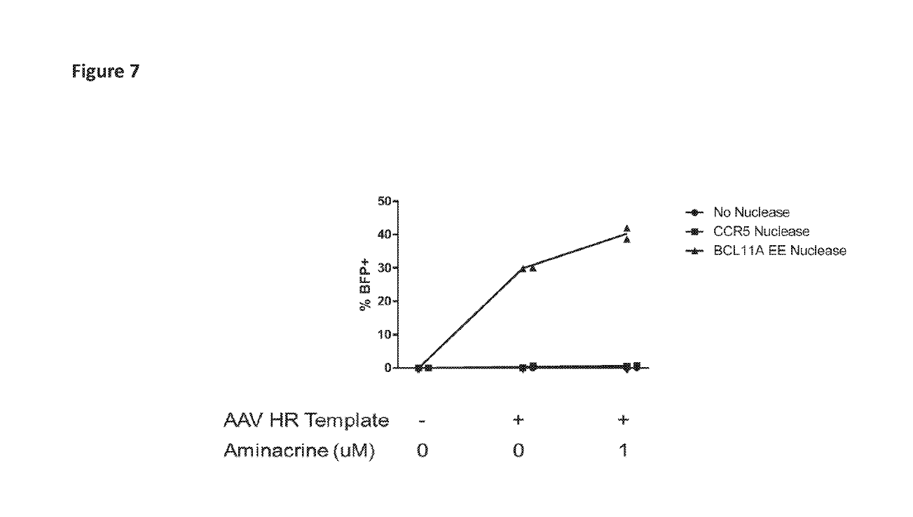

To determine whether treatment with aminacrine could yield elevated HDR rates in methylcellulose colonies derived from primary human hematopoietic stem and progenitor cells, colonies were harvested and analyzed by flow cytometry. Aminacrine treatment increased the proportion of cells undergoing HDR as determined by flow cytometry in samples treated with both megaTAL and rAAV vector. FIG. 7.

### Example 6

### Aminacrine Increases HDR in Bulk HSC Population

Primary human peripheral blood-derived CD34+ cells were cultured in cytokine-supplemented media for 48 hours. Cells were washed and electroporated with an mRNA encoding a megaTAL specific for the human BCL11A erythroid enhancer region. Cells were then incubated with AAV vector with or without aminacrine at a concentration of 1.0 μM for 24 hours, washed and cultured in cytokine-supplemented media. HDR was analyzed at regular timepoints by flow cytometry to determine BFP fluorescence.

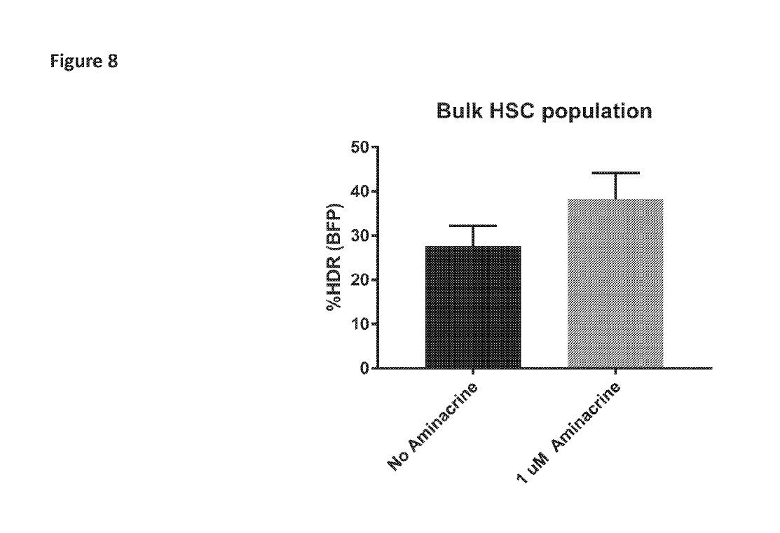

Aminacrine increased HDR frequency in CD34+ cells at the BCL11A locus (% BFP+ cells). FIG. 8.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A composition comprising a population of cells, a genome editing enhancer, and an engineered nuclease, and/or optionally, an mRNA encoding the engineered nuclease.

2. A composition comprising a population of cells, a genome editing enhancer, a donor repair template, and an engineered nuclease, and/or optionally, an mRNA encoding the engineered nuclease.

3. The composition of claim 1 or claim 2, wherein the population of cells comprises stem cells.

4. The composition of any one of claims 1 to 3, wherein the the population of cells comprises hematopoietic cells.

5. The composition of any one of claims 1 to 4, wherein the population of cells comprises CD34⁺ cells, CD133⁺ cells, CD34⁺CD133⁺ cells, or CD34⁺CD38^{Lo}CD90⁺CD45RA⁻ cells.

6. The composition of claim 1 or claim 2, wherein the population of cells comprises immune effector cells.

7. The composition of claim 1 or claim 2, wherein the population of cells comprises CD3⁺, CD4⁺, CD8⁺ cells, or a combination thereof.

8. The composition of claim 1 or claim 2, wherein population of cells comprises T cells.

9. The composition of claim 1 or claim 2, wherein the population of cells comprises cytotoxic T lymphocytes (CTLs), a tumor infiltrating lymphocytes (TILs), or a helper T cells.

10. The composition of claim 1 or claim 2, wherein the source of the cells is peripheral blood mononuclear cells, bone marrow, lymph nodes tissue, cord blood, thymus issue, tissue from a site of infection, ascites, pleural effusion, spleen tissue, or tumors.

11. A composition comprising a genome editing enhancer, and an engineered nuclease, and/or optionally, an mRNA encoding the engineered nuclease.

12. A composition comprising a genome editing enhancer, a donor repair template, and an engineered nuclease, and/or optionally, an mRNA encoding the engineered nuclease.

13. The composition of any one of claims 1 to 12, wherein the genome editing enhancer is a DNA intercalator.

14. The composition of any one of claims 1 to 13, wherein the genome editing enhancer is selected from the group consisting of: a monofunctional DNA intercalator, a bifunctional DNA intercalator, or a polyfunctional DNA intercalator.

15. The composition of any one of claims 1 to 14, wherein the genome editing enhancer is selected from the group consisting of: acridines, anthracyclines, alkaloids, coumarins, and phenanthridines.

16. The composition of any one of claims 1 to 15, wherein the genome editing enhancer is selected from the group consisting of: 1,8-naphthalimide, 4′6-diamidino-α-phenylindole, acridines, acridine orange, acriflavine, acronycine, actinodaphnidine, aminacrine, amsacrine, anthracycline, anthramycin, anthrapyrazole, benzophenanthridine alkaloids, berbamine, berberine, berberrubine, bleomycin, BOBO-1, BOBO-3, boldine, BO-PRO-1, BO-PRO-3, bublocapnine, camptothecin, cassythine, chartreusin, chloroquine, chromomycin, cinchonidine, cinchonine, coptisine, coralyne, coumarin, cryptolepine, dactinomycin, DAPI, daunorubicin, dicentrine, dictamine, distamycin, doxorubicin, ellipticine, emetine, ethacridine, ethidium, evolitrine, fagarine, fagaronine, fluorcoumanin, GelStar, gentamicin, glaucine, harmaline, harmine, harmine, hedamycin, hexidium, Hoechst 33258, Hoechst 33342, homidium, hycanthone, imidazoacridinone, an indazole analog, iodide, isocorydine, isoquinoline alkaloids, jatrorrhizine, JOJO-1, JO-PRO-1, kinetin riboside, kokusainine, lobeline, LOLO-1, LO-PRO-1, lucanthone, masculine, matadine, mepacrine, a metallo-intercalator, mithramycin, mitoxantrone, neocryptolepine, netropsin, nitidine, nitracrine, nogalamycin, norharman, OliGreen, palmatine, phenanthridine, PicoGreen, pirarubicin, polypyridyls, POPO-1, POPO-3, PO-PRO-1, PO-PRO-3, proflavine, propidium, psoralen, quinacrine, quinidine, quinine, quinoxalines, RiboGreen, a rhodium based intercalator, a ruthenium based intercalator, sanguinarine, serpentine, skimmianine, streptomycin, SYBR DX, SYBR Gold, SYBR Green I, SYBR Green II, SYTO-11, SYTO-12, SYTO-13, SYTO-14, SYTO-15, SYTO-16, SYTO-17, SYTO-20, SYTO-21, SYTO-22, SYTO-23, SYTO-24, SYTO-25, SYTO-40, SYTO-41, SYTO-42, SYTO-43, SYTO-44, SYTO-45, SYTO-59, SYTO-60, SYTO-61, SYTO-62, SYTO-63, SYTO-64, SYTO-80, SYTO-81, SYTO-82, SYTO-83, SYTO-84, SYTO-85, SYTOX blue, SYTOX green, SYTOX orange, tacrine, thalidomide, thiazole orange, tilorone, TO-PRO-1, TO-PRO-3, TO-PRO-5, TOTO-1, TOTO-3, usambarensine, YO-PRO-1, YO-PRO-3, YOYO-1, YOYO-3, and analogs and derivatives thereof.

17. The composition of any one of claims 1 to 16, wherein the genome editing enhancer is selected from the group consisting of: tilorone, aminacrine, homidium bromide (ethidium bromide), harmine, hycanthone, daunorubicin, sanguinarine sulfate, kinetin riboside, ethacridine lactate, and cyclohexamide.

18. The composition of any one of claims 1 to 17, wherein the genome editing enhancer is selected from the group consisting of: tilorone, aminacrine, homidium bromide (ethidium bromide), and harmine.

19. The composition of any one of claims 1 to 18, wherein the genome editing enhancer is an acridine or diacridine.

20. The composition of any one of claims 1 to 19, wherein the genome editing enhancer is aminacrine (9-aminoacridine).

21. A composition comprising a cell, an acridine, and an engineered nuclease, and/or optionally, an mRNA encoding the engineered nuclease.

22. A composition comprising a cell, an acridine, a donor repair template, and an engineered nuclease, and/or optionally, an mRNA encoding the engineered nuclease.

23. A composition comprising a cell, 9-aminoacridine, and an engineered nuclease, and/or optionally, an mRNA encoding the engineered nuclease.

24. A composition comprising a cell, 9-aminoacridine, a donor repair template, and an engineered nuclease, and/or optionally, an mRNA encoding the engineered nuclease.

25. A composition comprising an acridine, and an engineered nuclease, and/or optionally, an mRNA encoding the engineered nuclease.

26. A composition comprising an acridine, a donor repair template, and an engineered nuclease, and/or optionally, an mRNA encoding the engineered nuclease.

27. A composition comprising 9-aminoacridine, and an engineered nuclease, and/or optionally, an mRNA encoding the engineered nuclease.

28. A composition comprising 9-aminoacridine, a donor repair template, and an engineered nuclease, and/or optionally, an mRNA encoding the engineered nuclease.

29. The composition of any one of claims 1-28, wherein the engineered nuclease is selected from the group consisting of: a meganuclease, a megaTAL, a TALEN, a ZFN, or a CRISPR/Cas nuclease.

30. The composition of claim 29, wherein the meganuclease is engineered from an LAGLIDADG homing endonuclease (LHE) selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

31. The composition of claim 29 or claim 30, wherein the meganuclease is engineered from an LHE selected from the group consisting of: I-CpaMI, I-HjeMI, I-OnuI, I-PanMI, and SmaMI.

32. The composition of any one of claims 29-31, wherein the meganuclease is engineered from an I-OnuI LHE.

33. The composition of claim 29, wherein the megaTAL comprises a TALE DNA binding domain and an engineered meganuclease.

34. The composition of claim 33, wherein the TALE binding domain comprises about 9.5 TALE repeat units to about 11.5 TALE repeat units.

35. The composition of claim 33 or claim 34, wherein the meganuclease is engineered from an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

36. The composition of any one of claims 33-35, wherein the meganuclease is engineered from an LHE selected from the group consisting of: I-CpaMI, I-HjeMI, I-OnuI, I-PanMI, and SmaMI.

37. The composition of any one of claims 33-36, wherein the meganuclease is engineered from an I-OnuI LHE.

38. The composition of claim 29, wherein the TALEN comprises a TALE DNA binding domain and an endonuclease domain or half-domain.

39. The composition of claim 38, wherein the TALE DNA binding domain comprises about 9.5 TALE repeat units to about 11.5 TALE repeat units.

40. The composition of claim 38 or claim 39, wherein the endonuclease domain is isolated from a type-II restriction endonuclease.

41. The composition of any one of claims 38-40, wherein the endonuclease domain is isolated from FokI.

42. The composition of claim 39, wherein the ZFN comprises a zinc finger DNA binding domain and an endonuclease domain or half-domain.

43. The composition of claim 42, wherein the zinc finger DNA binding domain comprises 2, 3, 4, 5, 6, 7, or 8 zinc finger motifs.

44. The composition of claim 42 or claim 43, wherein the ZFN comprises a TALE binding domain.

45. The composition of claim 44, wherein the TALE DNA binding domain comprises about 9.5 TALE repeat units to about 11.5 TALE repeat units.

46. The composition of any one of claims 42-45, wherein the endonuclease domain is isolated from a type-II restriction endonuclease.

47. The composition of any one of claims 42-46, wherein the endonuclease domain is isolated from FokI.

48. The composition of claim 39, wherein the engineered nuclease comprises a CRISPR/Cas nuclease.

49. The composition of claim 48, wherein the Cas nuclease is Cas9 or Cpf1.

50. The composition of claim 48 or claim 49, wherein the Cas nuclease further comprises one or more TALE DNA binding domains.

51. The composition of any one of claims 48-50, wherein the composition further comprises a tracrRNA, and one or more crRNAs that target a protospacer sequence in the genome of the cell.

52. The composition of any one of claims 48-50, wherein the composition further comprises one or more sgRNAs that target a protospacer sequence in the genome of the cell.

53. The composition of any one of claims 1 to 52, wherein the engineered nuclease comprises an end-processing enzymatic activity.

54. The composition of claim 53, wherein the end-processing enzymatic activity is 5-3′ exonuclease, 5-3′ alkaline exonuclease, 3-5′exonuclease, 5′ flap endonuclease, helicase or template-independent DNA polymerases activity.

55. The composition of claim 54, wherein the end-processing enzymatic activity is 3-5′exonuclease activity of Trex2 or a biologically active fragment thereof.

56. The composition of any one of claims 1 to 52, wherein the composition further comprises an end-processing enzyme, or an mRNA encoding the end-processing enzyme.

57. The composition of claim 56, wherein the end-processing enzyme exhibits 5-3′ exonuclease, 5-3′ alkaline exonuclease, 3-5′exonuclease, 5′ flap endonuclease, helicase or template-independent DNA polymerases activity.

58. The composition of claim 56 or claim 57, wherein the end-processing enzyme comprises Trex2 or a biologically active fragment thereof.

59. The composition of any one of claims 1 to 58, comprising a donor repair template that encodes: β globin, δ globin, γ globin, BCL11A, KLF1, CCR5, CXCR4, PPP1R12C (AAVS1), HPRT, albumin, Factor VIII, Factor IX, LRRK2, Htt, SOD1, C9orf72, TARDBP, FUS, RHO, CFTR, SFTPB, TRAC, TRBC, PD1, CTLA-4, HLA A, HLA B, HLA C, HLA-DP, HLA-DQ, HLA-DR, LMP7, TAP 1, TAP2, TAPBP, CIITA, DMD, GR, IL2RG, Rag-1, RFX5, FAD2, FAD3, ZP15, KASII, MDH, EPSPS, or a fragment thereof.

60. The composition of any one of claims 1 to 58, comprising a donor repair template that encodes a bispecific T cell engager (BiTE) molecule; a hormone; a cytokine (e.g., IL-2, insulin, IFN-γ, IL-7, IL-21, IL-10, IL-12, IL-15, and TNF-α), a chemokine (e.g., MIP-1α, MIP-1β, MCP-1, MCP-3, and RANTES), a cytotoxin (e.g., Perforin, Granzyme A, and Granzyme B), a cytokine receptor (e.g., an IL-2 receptor, an IL-7 receptor, an IL-12 receptor, an IL-15 receptor, and an IL-21 receptor), or an engineered antigen receptor.

61. The composition of any one of claims 1 to 58, comprising a donor repair template that encodes an engineered T cell receptor (TCR), a chimeric antigen receptor (CAR), a Daric receptor or components thereof, or a chimeric cytokine receptor.

62. A method of increasing genome editing in a population of cells comprising:
(a) introducing an engineered nuclease into a population of cells; and
(b) contacting the population of cells with a genome editing enhancer,
wherein expression of the engineered nuclease in the presence of the genome editing enhancer increases the frequency of genome editing in the population of cells.

63. A method of increasing homology directed repair (HDR) in a population of cells comprising:
(a) contacting the population of cells with a genome editing enhancer;
(b) introducing an engineered nuclease to generate a double-strand break (DSB) at a target site; and
(b) introducing a donor repair template into the population of cells;
wherein expression of the engineered nuclease in the presence of the genome editing enhancer and the donor repair template increases the frequency of incorporation of the donor repair template at the target site by homology directed repair (HDR).

64. A method of increasing non-homologous end joining (NHEJ) in a population of cells comprising:
(a) contacting the population of cells with a genome editing enhancer;
(b) introducing an engineered nuclease to generate a double-strand break (DSB) at a target site;
(a) introducing an engineered nuclease into a population of cells; and
wherein expression of the engineered nuclease in the presence of the genome editing enhancer increases the frequency of NHEJ at the target site.

65. The method of any one of claims 62-64, wherein the cell is a hematopoietic cell.

66. The method of any one of claims 62-65, wherein the cell is an immune effector cell.

67. The method of any one of claims 62-66, wherein the cell is CD3⁺, CD4⁺, CD8⁺, or a combination thereof.

68. The method of any one of claims 62-67, wherein the cell is a T cell.

69. The method of any one of claims 62-68, wherein the cell is a cytotoxic T lymphocyte (CTL), a tumor infiltrating lymphocyte (TIL), or a helper T cell.

70. The method of any one of claims 62-69, wherein the source of the cell is peripheral blood mononuclear cells, bone marrow, lymph nodes tissue, cord blood, thymus issue, tissue from a site of infection, ascites, pleural effusion, spleen tissue, or tumors.

71. The method of any one of claims 62-65, wherein the cell is a hematopoietic stem cell or hematopoietic progenitor cell.

72. The method of any one of claims 62-65, wherein the cell is a CD34⁺ cell.

73. The method of any one of claims 62-65, wherein the cell is a CD133⁺ cell.

74. The method of any one of claims 62-65, wherein the cell is a CD34⁺CD38^{Lo}CD90⁺CD45RA⁻ cell.

75. The method of any one of claims 62-74, wherein the genome editing enhancer is a DNA intercalator.

76. The method of any one of claims 62-75, wherein the genome editing enhancer is selected from the group consisting of: a monofunctional DNA intercalator, a bifunctional DNA intercalator, or a polyfunctional DNA intercalator.

77. The method of any one of claims 62-76, wherein the genome editing enhancer is selected from the group consisting of: acridines, anthracyclines, alkaloids, coumarins, and phenanthridines.

78. The method of any one of claims 62-77, wherein the genome editing enhancer is selected from the group consisting of: 1,8-naphthalimide, 4′6-diamidino-α-phenylindole, acridines, acridine orange, acriflavine, acronycine, actinodaphnidine, aminacrine, amsacrine, anthracycline, anthramycin, anthrapyrazole, benzophenanthridine alkaloids, berbamine, berberine, berberrubine, bleomycin, BOBO-1, BOBO-3, boldine, BO-PRO-1, BO-PRO-3, bublocapnine, camptothecin, cassythine, chartreusin, chloroquine, chromomycin, cinchonidine, cinchonine, coptisine, coralyne, coumarin, cryptolepine, dactinomycin, DAPI, daunorubicin, dicentrine, dictamine, distamycin, doxorubicin, ellipticine, emetine, ethacridine, ethidium, evolitrine, fagarine, fagaronine, fluorcoumanin, GelStar, gentamicin, glaucine, harmaline, harmine, harmine, hedamycin, hexidium, Hoechst 33258, Hoechst 33342, homidium, hycanthone, imidazoacridinone, an indazole analog, iodide, isocorydine, isoquinoline alkaloids, jatrorrhizine, JOJO-1, JO-PRO-1, kinetin riboside, kokusainine, lobeline, LOLO-1, LO-PRO-1, lucanthone, masculine, matadine, mepacrine, a metallo-intercalator, mithramycin, mitoxantrone, neocryptolepine, netropsin, nitidine, nitracrine, nogalamycin, norharman, OliGreen, palmatine, phenanthridine, PicoGreen, pirarubicin, polypyridyls, POPO-1, POPO-3, PO-PRO-1, PO-PRO-3, proflavine, propidium, psoralen, quinacrine, quinidine, quinine, quinoxalines, RiboGreen, a rhodium based intercalator, a ruthenium based intercalator, sanguinarine, serpentine, skimmianine, streptomycin, SYBR DX, SYBR Gold, SYBR Green I, SYBR Green II, SYTO-11, SYTO-12, SYTO-13, SYTO-14, SYTO-15, SYTO-16, SYTO-17, SYTO-20, SYTO-21, SYTO-22, SYTO-23, SYTO-24, SYTO-25, SYTO-40, SYTO-41, SYTO-42, SYTO-43, SYTO-44, SYTO-45, SYTO-59, SYTO-60, SYTO-61, SYTO-62, SYTO-63, SYTO-64, SYTO-80, SYTO-81, SYTO-82, SYTO-83, SYTO-84, SYTO-85, SYTOX blue, SYTOX green, SYTOX orange, tacrine, thalidomide, thiazole orange, tilorone, TO-PRO-1, TO-PRO-3, TO-PRO-5, TOTO-1, TOTO-3, usambarensine, YO-PRO-1, YO-PRO-3, YOYO-1, YOYO-3, and analogs and derivatives thereof.

79. The method of any one of claims 62-78, wherein the genome editing enhancer is selected from the group consisting of: tilorone, aminacrine, homidium bromide (ethidium bromide), harmine, hycanthone, daunorubicin, sanguinarine sulfate, kinetin riboside, ethacridine lactate, and cyclohexamide.

80. The method of any one of claims 62-79, wherein the genome editing enhancer is selected from the group consisting of: tilorone, aminacrine, homidium bromide (ethidium bromide), and harmine.

81. The method of any one of claims 62-80, wherein the genome editing enhancer is an acridine or diacridine.

82. The method of any one of claims 62-81, wherein the genome editing enhancer is aminacrine (9-aminoacridine).

83. The method of any one of claims 62-82, wherein the engineered nuclease is selected from the group consisting of: a meganuclease, a megaTAL, a TALEN, a ZFN, or a CRISPR/Cas nuclease.

84. The method of claim 83, wherein the meganuclease is engineered from an LAGLIDADG homing endonuclease (LHE) selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

85. The method of any one of claim 83 or claim 84, wherein the meganuclease is engineered from an LHE selected from the group consisting of: I-CpaMI, I-HjeMI, I-OnuI, I-PanMI, and SmaMI.

86. The method of any one of claims 84-85, wherein the meganuclease is engineered from an I-OnuI LHE.

87. The method of claim 83, wherein the megaTAL comprises a TALE DNA binding domain and an engineered meganuclease.

88. The method of claim 87, wherein the TALE binding domain comprises about 9.5 TALE repeat units to about 11.5 TALE repeat units.

89. The method of claim 87 or claim 88, wherein the meganuclease is engineered from an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

90. The method of any one of claims 87-89, wherein the meganuclease is engineered from an LHE selected from the group consisting of: I-CpaMI, I-HjeMI, I-OnuI, I-PanMI, and SmaMI.

91. The method of any one of claims 87-90, wherein the meganuclease is engineered from an I-OnuI LHE.

92. The method of claim 83, wherein the TALEN comprises a TALE DNA binding domain and an endonuclease domain or half-domain.

93. The method of claim 92, wherein the TALE DNA binding domain comprises about 9.5 TALE repeat units to about 11.5 TALE repeat units.

94. The method of claim 92-93, wherein the endonuclease domain is isolated from a type-II restriction endonuclease.

95. The method of any one of claims 92-94, wherein the endonuclease domain is isolated from FokI.

96. The method of claim 83, wherein the ZFN comprises a zinc finger DNA binding domain and an endonuclease domain or half-domain.

97. The method of claim 96, wherein the zinc finger DNA binding domain comprises 2, 3, 4, 5, 6, 7, or 8 zinc finger motifs.

98. The method of claim 96 or claim 97, wherein the ZFN comprises a TALE binding domain.

99. The method of claim 98, wherein the TALE DNA binding domain comprises about 9.5 TALE repeat units to about 11.5 TALE repeat units.

100. The method of any one of claims 96-99, wherein the endonuclease domain is isolated from a type-II restriction endonuclease.

101. The method of any one of claims 96-100, wherein the endonuclease domain is isolated from FokI.

102. The method of claim 83, wherein the engineered nuclease comprises a CRISPR/Cas nuclease.

103. The method of claim 102, wherein the Cas nuclease is Cas9 or Cpf1.

104. The method of claim 102 or claim 103, wherein the Cas nuclease further comprises one or more TALE DNA binding domains.

105. The method of any one of claims 102-104, wherein the composition further comprises a tracrRNA, and one or more crRNAs that target a protospacer sequence in the genome of the cell.

106. The method of any one of claims 102-104, wherein the composition further comprises one or more sgRNAs that target a protospacer sequence in the genome of the cell.

107. The method of any one of claims 62 to 106, wherein the engineered nuclease comprises an end-processing enzymatic activity.

108. The method of claim 107, wherein the end-processing enzymatic activity is 5-3′ exonuclease, 5-3′ alkaline exonuclease, 3-5′exonuclease, 5′ flap endonuclease, helicase or template-independent DNA polymerases activity.

109. The method of claim 108, wherein the end-processing enzymatic activity is 3-5′exonuclease activity of Trex2 or a biologically active fragment thereof.

110. The method of any one of claims 62-106, wherein the composition further comprises an end-processing enzyme, or an mRNA encoding the end-processing enzyme.

111. The method of claim 110, wherein the end-processing enzyme exhibits 5-3′ exonuclease, 5-3′ alkaline exonuclease, 3-5′exonuclease, 5′ flap endonuclease, helicase or template-independent DNA polymerases activity.

112. The method of claim 110 or claim 111, wherein the end-processing enzyme comprises Trex2 or a biologically active fragment thereof.

113. The method of any one of claims 62 to 112, comprising a donor repair template that encodes: β globin, δ globin, γ globin, BCL11A, KLF1, CCR5, CXCR4, PPP1R12C (AAVS1), HPRT, albumin, Factor VIII, Factor IX, LRRK2, Htt, SOD1, C9orf72, TARDBP, FUS, RHO, CFTR, SFTPB, TRAC, TRBC, PD1, CTLA-4, HLA A, HLA B, HLA C, HLA-DP, HLA-DQ, HLA-DR, LMP7, TAP 1, TAP2, TAPBP, CIITA, DMD, GR, IL2RG, Rag-1, RFX5, FAD2, FAD3, ZP15, KASII, MDH, EPSPS, or a fragment thereof.

114. The method of any one of claims 62 to 112, comprising a donor repair template that encodes: a bispecific T cell engager (BiTE) molecule; a hormone; a cytokine (e.g., IL-2, insulin, IFN-γ, IL-7, IL-21, IL-10, IL-12, IL-15, and TNF-α), a chemokine (e.g., MIP-1α, MIP-1β, MCP-1, MCP-3, and RANTES), a cytotoxin (e.g., Perforin, Granzyme A, and Granzyme B), a cytokine receptor (e.g., an IL-2 receptor, an IL-7 receptor, an IL-12 receptor, an IL-15 receptor, and an IL-21 receptor), or an engineered antigen receptor.

115. The method of any one of claims 62 to 112, comprising a donor repair template that encodes: an engineered T cell receptor (TCR), a chimeric antigen receptor (CAR), a Daric receptor or components thereof, or a chimeric cytokine receptor.

116. A cell produced by a method of any one of claims 62 to 115.

117. A composition comprising a cell according to claim 116.

118. A pharmaceutical composition comprising a pharmaceutically acceptable carrier and a cell according to claim 116.
